(19) United States
(12) Patent Application Publication
(10) Pub. No.: US 2025/0311936 A1
(43) Pub. Date: Oct. 9, 2025

(54) CONTINUOUS BLOOD FLOW VISUALIZATION WITH LASER SPECKLE CONTRAST IMAGING

(71) Applicant: BOARD OF REGENTS, THE UNIVERSITY OF TEXAS SYSTEM, Austin, TX (US)

(72) Inventors: Andrew DUNN, Austin, TX (US); David MILLER, Austin, TX (US); Colin SULLENDER, Harrison, NJ (US)

(21) Appl. No.: 18/860,498

(22) PCT Filed: Apr. 27, 2023
(86) PCT No.: PCT/US2023/020197

§ 371 Date: Oct. 25, 2024

Related U.S. Application Data
(60) Provisional application No. 63/335,854, filed on Apr. 28, 2022.

(51) Int. Cl. A61B 5/026; A61B 5/00; A61B 5/0275; G06T 7/33; G06T 11/00; G16H 20/40; G16H 30/40

(52) U.S. Cl. CPC A61B 5/0261; A61B 5/0042; A61B 5/0275; G06T 7/337; G06T 11/00; G16H 20/40; G16H 30/40; A61B 2505/05; A61B 2576/026; G06T 2200/24; G06T 2207/30016; G06T 2207/30104; G06T 2210/41

(57) ABSTRACT

A system and method for visualizing blood flow are disclosed. The method generally includes obtaining a laser speckle contrast imaging (LSCI) image of blood flow; obtaining a white light image of a tissue, the white light image capturing an anatomical structure of a subject in a region associated with the LSCI image of the blood flow; spatially registering the LSCI image and the white light image with one another; overlaying the spatially registered LSCI and white light images; and generating display data which includes the spatially registered LSCI and white light images and that continuously depicts the blood flow overlaying the tissue.

### CROSS-REFERENCE TO RELATED APPLICATIONS

This application claims the benefit of and priority to U.S. Provisional Patent App. No. 63/335,854, filed Apr. 28, 2022, which is incorporated herein by reference in its entirety.

### STATEMENT REGARDING FEDERALLY FUNDED RESEARCH

This invention was made with government support under Grant No. R01 EB011556 awarded by the National Institutes of Health. The government has certain rights in the invention.

### BACKGROUND

Cerebral blood flow (CBF) monitoring is routine during cerebrovascular surgery to inform decision making. In cerebral aneurysm clipping cases, various technologies are routinely used to confirm patency in vessels and determine successful aneurysmal obliteration. Current intraoperative tools for CBF monitoring and visualization include indocyanine green angiography (ICGA), doppler, and transit-time ultrasound, and percutaneous transfemoral digital subtraction angiography (DSA). ICGA records the fluorescence wash in of a bolus of indocyanine green after intravenous injection. DSA images are acquired by obtaining multiple time-controlled X-rays as contrast medium is injected intra-arterially.

### SUMMARY

One implementation of the present disclosure is a method for visualizing blood flow, the method including: obtaining a laser speckle contrast imaging (LSCI) image of blood flow; obtaining a white light image of a tissue, the white light image capturing an anatomical structure of a subject in a region associated with the LSCI image of the blood flow; spatially registering the LSCI image and the white light image with one another; overlaying the spatially registered LSCI and white light images; and generating display data that continuously depicts the blood flow overlaying the tissue, wherein the display data includes the spatially registered LSCI and white light images.

In some implementations, the display data continuously depicts the blood flow overlaying the tissue in real-time during a surgical procedure.

In some implementations, the method further includes displaying the display data on a user interface.

In some implementations, the tissue includes vasculature.

In some implementations, the tissue is brain tissue.

In some implementations, spatially registering the LSCI image and the white light image with one another includes creating a lookup table based on a spatial transformation used to register the LSCI and white light images.

In some implementations, a overlaying the spatially registered LSCI and white light images includes mapping respective pixels from the LSCI image to respective pixels from the white light image using the lookup table.

In some implementations, overlaying the spatially registered LSCI and white light images further includes contrast stretching the LSCI image, mapping the LSCI image to an n-bit color map, and performing a weighted sum with the white light image.

Another implementation of the present disclosure a system for blood flow visualization, the system including: a first light source configured to illuminate blood flow at a target region of a subject; a first camera configured to record a raw laser speckle image of the blood flow; and a computing device including a processor and a memory, the memory having instructions stored thereon that, when executed by the processor, cause the computing device to: obtain, via the first camera, the raw laser speckle image of the blood flow; derive a laser speckle contrast imaging (LSCI) image from the raw laser speckle image; obtain a white light image of tissue at the target region of the subject, the white light image capturing an anatomical structure; spatially register the LSCI image and the white light image with one another; overlay the spatially registered LSCI and white light images; and generate display data that continuously depicts the blood flow overlaying the tissue, wherein the display data includes the spatially registered LSCI and white light images.

In some implementations, the system is a surgical microscope, an endoscope, an exoscope, a robotic surgery platform, a stand-alone imaging system, or system dedicated for blood flow imaging.

In some implementations, the display data continuously depicts the blood flow overlaying the tissue in real-time during a surgical procedure.

In some implementations, the instructions further cause the computing device to present the display data on a user interface.

In some implementations, the tissue includes vasculature.

In some implementations, the tissue is brain tissue.

In some implementations, spatially registering the LSCI image and the white light image with one another includes creating a lookup table based on a spatial transformation used to register the LSCI and white light images.

In some implementations, overlaying the spatially registered LSCI and white light images includes mapping respective pixels from the LSCI image to respective pixels from the white light image using the lookup table.

In some implementations, overlaying the spatially registered LSCI and white light images further includes contrast stretching the LSCI image, mapping the LSCI image to an n-bit color map, and performing a weighted sum with the white light image.

Yet another implementation of the present disclosure a method for visualizing blood flow, the method including continuously: capturing, using a first image capture device, a laser speckle contrast imaging (LSCI) image of blood flow in a subject; capturing, using a second image capture device, a white light image of a tissue, the white light image capturing an anatomical structure; co-registering the LSCI image and the white light image; overlaying the co-registered LSCI and white light images; and displaying, via a user interface, the overlayed and co-registered LSCI and white light images.

In some implementations, the tissue includes vasculature or brain tissue.

In some implementations, co-registering the LSCI image and the white light image with one another includes creating a lookup table based on a spatial transformation used to register the LSCI and white light images.

### BRIEF DESCRIPTION OF THE DRAWINGS

FIG. 1 is a block diagram of a blood flow visualization system, according to some implementations.

FIGS. 2A and 2B are diagrams of various configurations of the visualization system of FIG. 1, according to some implementations.

FIG. 3 is an image of an example configuration of the visualization system of FIG. 1, according to some implementations.

FIG. 4 is a block diagram of a computer system for implementing the processes described herein, according to some implementations.

FIG. 5 is a flow diagram of a method for visualizing blood flow, according to some implementations.

FIG. 6 is a flow diagram of a method for visualizing blood flow, according to some implementations.

FIGS. 7A-7D is an example image processing pipeline for generating an overlay of laser speckle contrast imaging (LSCI), according to some implementations. LCSI, thresholded, white light, and LCSI overlay images are shown in FIGS. 7A-7D, respectively.

FIG. 8 includes multiple example images captured from a patient before aneurysm clipping (e.g., pre-clipping), immediately after aneurysm clipping (e.g., post-clipping), and during indocyanine green angiography (ICGA), according to some implementations.

FIGS. 9A and 9B are example graphs of relative cerebral blood flow (CBF) during a clipping procedure, according to some implementations.

FIG. 10 includes multiple example images captured from a patient during ICGA, according to some implementations.

FIGS. 11A and 11B include example images that compare LSCI overlay (FIG. 11A) with ICGA overlay (FIG. 11B), according to some implementations.

FIG. 12A includes multiple example images of an arteriovenous malformation (AVM) resection, according to some implementations.

FIG. 12B is an example graph of averaged speckle contrast values before and after the AVM resection, according to some implementations.

FIG. 13A includes multiple example images illustrating a fluorescence intensity change during bolus administration of indocyanine green (ICG) and the LSCI measures of relative blood flow for the same regions of interest, according to some implementations.

FIG. 13B is an example graph of fluorescence intensity change during bolus administration of ICG and the LSCI measures of relative blood flow for the same regions of interest, according to some implementations.

Various objects, aspects, and features of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

Monitoring of cerebral blood flow (CBF) plays an important role in a myriad of neurosurgical and neuroscience applications, such as during cerebrovascular surgery. As mentioned above, current techniques are often insufficient for activity monitoring CBF in real or near-real time, as many techniques result only in period measurements or may require significant additional effort by physicians to implement. Thus, it may be generally more desirable for CBF to be continuously monitored, as opposed to periodically measured. As mentioned above, ICGA is an effective decision-making aid; however, it cannot provide continuous imaging as it requires an injected contrast agent. Doppler ultrasound provides absolute flow velocities, but is limited to measurement at single locations, and requires contact with the vessel of interest. DSA has been utilized for confirming aneurysmal occlusion and patency of the underlying parent vasculature; however, it is invasive and time-consuming relative to ICGA or doppler ultrasound, e.g., usually requiring removal of the surgical microscope, fluoroscopy, and transfemoral selective arterial catheterization.

Laser speckle contrast imaging (LSCI) has emerged as a promising tool to non-invasively monitor CBF because it produces real-time, full-field blood flow maps without any contrast agents. LSCI may therefore provide a potential continuous CBF monitoring solution. Several studies have demonstrated LSCI during neurosurgical procedures in humans and shown its promise as a CBF monitoring tool, including surgical revascularization, awake functional mapping, brain tumor resection, cortical spreading depression, and infarction during ischemic stroke. However, many previous clinical implementations required an external device to be introduced into the surgical field leading to disruptions of the surgical procedures. Other implementations incorporated the instrumentation into the neurosurgical operating microscope, eliminating the need for an external device and surgical disruption; however, the surgical procedure still would need to be paused while LSCI images were acquired, and it was not possible to record CBF images for long durations or simultaneously with ICGA.

An LSCI system and related methods are described herein that address these and other limitations of existing blood flow monitoring devices. In some implementations, the LSCI system described herein can be integrated into a surgical microscope or other medical device to facilitate real-time, continuous visualization of CBF overlayed onto the surgical field during surgical procedures, e.g., including simultaneous ICGA and LSCI imaging. To demonstrate effectiveness, the disclosed LSCI system and methods were evaluated during cerebral aneurysm clipping and arteriovenous malformation (AVM) resection surgeries. This disclosure demonstrates the potential of LSCI for human CBF monitoring in at least two ways: LSCI was performed continuously during cerebral aneurysm clipping and AVM resection surgeries without affecting the surgical workflow, including real-time visualization of CBF during aneurysm clip placement; and LSCI and ICGA were performed simultaneously to visualize CBF for five example neurovascular cases. Taken together, these results demonstrate that LSCI can monitor CBF continuously during neurovascular procedures when the LSCI device is integrated into the surgical microscope, and that LSCI and ICGA provide different yet complementary information about vessel perfusion.

### LSCI System

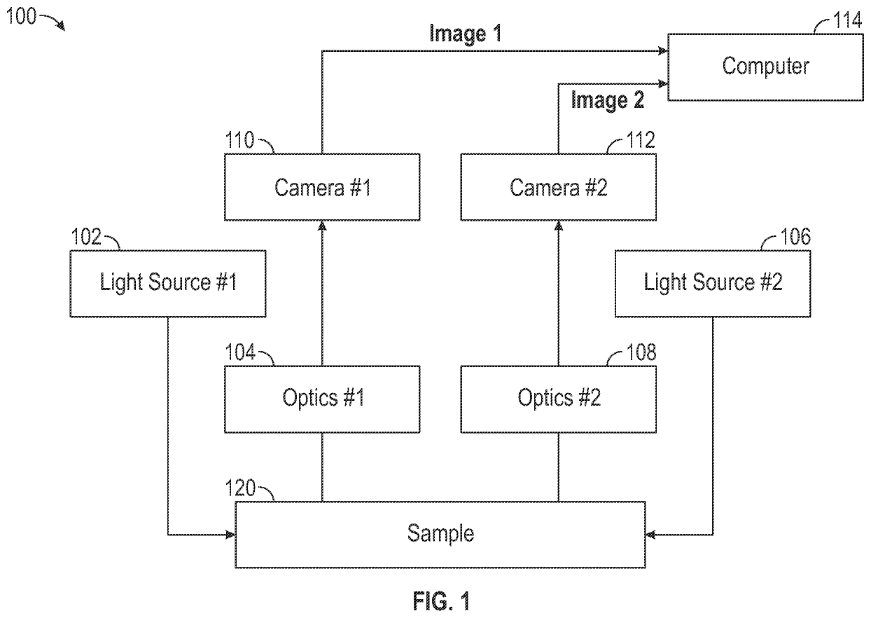

Referring now to FIG. 1, a block diagram of a blood flow visualization system 100 is shown, according to some implementations. System 100 is shown to include a first light source 102 and corresponding first optics 104, and a second light source 106 and corresponding second optics 108. In some implementations, first light source 102 and second light source 106 are configured to illuminate a target 120, e.g., a target area of a patient. At least a portion of the light emitted by first light source 102 and second light source 106 is then reflected off of target 120 and to a first camera 110 and a second camera 112. Each of first camera 110 and second camera 112 may then capture respective images of target 120 based on the reflected light and may transmit the images to a remote computing device 114.

As described herein, first light source 102 is generally any suitable light source that can produce and emit coherent light. As described herein, coherent light is generally light in which the electromagnetic waves maintain a fixed and predictable phase relationship with each other over a long enough period of time such that interference effects can be recorded with a sensor. Such light may include a single wavelength or narrow bandwidth. As those of the ordinary skill in the art will appreciate, coherent light does not practically require a single wavelength and industry standards allow for relative deviation, such as a bandwidth within +1 nm of a target wavelength. It is also possible to have a broad bandwidth such as if pulsed lasers are used as the coherent light source. In some implementations, the wavelength of first light source 102 is 785 nm (+5 nm) with a maximum output power of 300 mW. In some implementations, first light source 102 is a laser.

Second light source 106 is generally a source of white light or, alternatively, is generally configured to output light at a different wavelength that that of first light source 102. In some implementations, second light source 106 may be configured to emit light in the wavelength range of 350 nm to 850 nm. In this regard, the light emitted by second light source 106 may be a collection of multiple wavelengths or a single wavelength between 350 nm to 850 nm. As shown in FIG. 1, the light emitted by second light source 106 is generally directed towards 120 by second optics 108.

First optics 104 generally includes one or more optical components for filtering, focusing, and/or otherwise modifying the light emitted by first light source 102 and/or reflect off of target 120 from first light source 102. In some implementations, first optics 104 includes one or more lenses and/or mirrors for focusing and/or directing light from first light source 102 to target 120. In some implementations, first optics 104 includes one or more filters for filtering the light emitted by first light source 102 and reflected from target 120. In some implementations, first optics 104 includes one or more filters to reduce or prevent light from second light source 106 or other background light entering first camera 110. In some implementations, first optics 104 includes a band-pass filter centered on or around the wavelength of first light source 102. In some implementations, first optics 104 includes a polarizer to reduce specular reflection from target 120. In some such implementations, the polarizer may be on a rotatable mount that enables rotations of the polarizer.

Second optics 108 likewise generally includes one or more optical components for filtering, focusing, and/or otherwise modifying the light emitted by second light source 106 and/or reflected off of target 120 from second light source 106. In some implementations, second optics 108 includes one or more lenses and/or mirrors for focusing and/or directing light from second light source 106 to target 120. In some implementations, second optics 108 includes one or more filters for filtering the light emitted by second light source 106 and/or reflected off of target 120. In some implementations, second optics 108 includes one or more filters to reduce or prevent light from first light source 102 or other background light entering second camera 112. In some implementations, second optics 108 includes a band-pass filter centered on or around the wavelength of second light source 106. In some implementations, second optics 108 includes a polarizer to reduce specular reflection from target 120. In some such implementations, the polarizer may be on a rotatable mount that enables rotations of the polarizer.

While not shown in FIG. 1, it should be appreciated that first optics 104 and/or second optics 108 may include one or more components positioned between respective first light source 102 and second light source 106 and target 120, and/or may include one or more components positioned between target 120 and respective first camera 110 and second camera 112. In some implementations, first light source 102 and/or second light source 106 are fixedly coupled to one another to move in tandem with another, e.g., in response to manipulation by a user. For example, the user may move a surgical microscope and in so doing, will move first light source 102 and/or second light source 106 simultaneously with one another while maintaining both first camera 110 and second camera 112 in focus on target 120. In some implementations, first camera 110 and second camera 112 are in focus simultaneously. In some implementations, when first camera 110 is added to the microscope, optics for first camera 110 co-align it with second camera 112 such that the two cameras are in focus together.

First camera 110 and second camera 112 are generally any suitable image capturing devices. In some implementations, first camera 110 is specifically configured to capture images of target 120 in the wavelength range emitted by first light source 102. Likewise, in some implementations, second camera 112 can be specifically configured to capture images of target 120 in the wavelength range(s) emitted by second light source 106. In some implementations, second camera 112 is an imaging device that is coupled to, embedded in, or otherwise integrated with a medical imaging device. For example, second camera 112 may be associated with a surgical microscope, an endoscope, an exoscope, a robotic surgery platform, or the like.

In some implementations, first camera 110 is configured to capture raw LSCIs. In some implementations, first camera 110 is or includes a 10-bit or higher resolution charge coupled device (CCD) camera with exposure times ranging from about 1 ms to about 20 ms. In other implementations, first camera 110 may be or include a near-infrared (NIR)-enhanced complementary metal oxide semiconductor (CMOS) camera. In some implementations, first camera 110 and/or second camera 112 may operate at frame rates in the order of 10 to 160 frames per second; although, depending on the application, higher frame rates may be used. As described herein, the images captured by both first camera 110 and second camera 112 can include still images or can be recorded continuously to create a video.

In some implementations, although not shown in the figures, system 100 can further include a third camera for capturing ICGA images. In some such implementations, ICGA images are represented as raw fluorescence intensity images that were collected by a built-in fluorescence camera. In some implementations, the third camera may include a NIR camera. Such fluorescence imaging includes, for example, ICGA. In some implementations, include simultaneously performing laser speckle contrast imaging and additional imaging, a single camera may be used to record the fluorescence images and the raw laser speckle images by interleaving the image acquisitions for laser speckle and fluorescence. Additionally, or alternatively, in some implementations, a single camera could be used to perform the laser speckle contrast imaging, fluorescence imaging, and white light imaging as mentioned above.

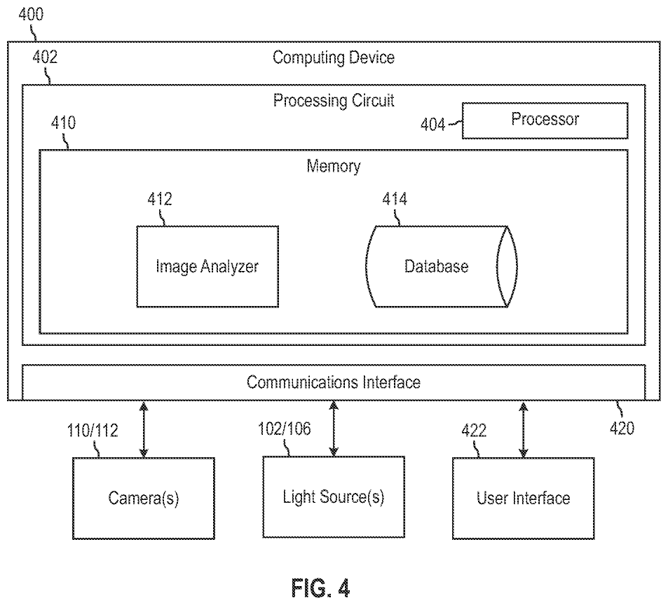

In general, remote computing device 114 is any computing device that is external to or remote from first camera 110 and/or second camera 112. The computing device 114 can be coupled to the first camera 110 and/or second camera 112 through one or more communication links. This disclosure contemplates the communication links are any suitable communication link. For example, a communication link may be implemented by any medium that facilitates data exchange between the network elements including, but not limited to, wired, wireless and optical links. Remote computing device 114 may generally include a processing circuit that includes a processor and memory, wherein the memory stores instructions for performing the various methods and processes described herein. Details of remote computing device 114 are provided below with respect to FIG. 4. As shown, remote computing device 114 is generally configured to receive images captured by first camera 110 and second camera 112, e.g., LSCIs and white-light images of target 120, for processing and/or storage. Additionally, in some implementations, remote computing device 114 may command the various other components of system 100 (e.g., first light source 102, second light source 106, first camera 110, and/or second camera 112) to capture images. For example, remote computing device 114 may be configured to activate one or both of first light source 102 and second light source 106 and can simultaneously operate first camera 110 and second camera 112 to capture images.

As discussed herein, it is generally difficult for surgeons to understand the anatomical structure in an LSCI image which shows blood flow. However, system 100 addresses this problem by overlaying a LSCI blood flow image-captured by first camera 110—on a second image that shows anatomical structure—captured by second camera 112. To overlay the LSCI image, the LSCI image captured by first camera 110 may be “thresholded,” or subject to thresholding, to show only flow between set values (e.g., “min” and “max”). In particular, remote computing device 114 may apply thresholding techniques to the image captured by first camera 110. Subsequently, remote computing device 114 spatially registers the thresholded LSCI image with the second image (e.g., the white-light image) captured by second camera 112 and then overlays the thresholded and registered LSCI image onto the second image.

As mentioned above, second camera 112 may be an image capture device coupled to or embedded in a medical imaging device. Accordingly, in various implementations, remote computing device 114 can be configured to: overlay an LSCI image captured by first camera 110 on an image from a surgical microscope with white light illumination or any wavelength that comprises white light from 350 nm to 850 nm, as captured by second camera 112; overlay an LSCI image captured by first camera 110 on an image from an endoscope with white light illumination or any wavelength that comprises white light from 350 nm to 850 nm, as captured by second camera 112; overlay an LSCI image captured by first camera 110 on an image from an exoscope with white light illumination or any wavelength that comprises white light from 350 nm to 850 nm, as captured by second camera 112; or overlay an LSCI image captured by first camera 110 on an image from a robot surgery platform with white light illumination or any wavelength that comprises white light from 350 nm to 850 nm, as captured by second camera 112.

### Example Configurations

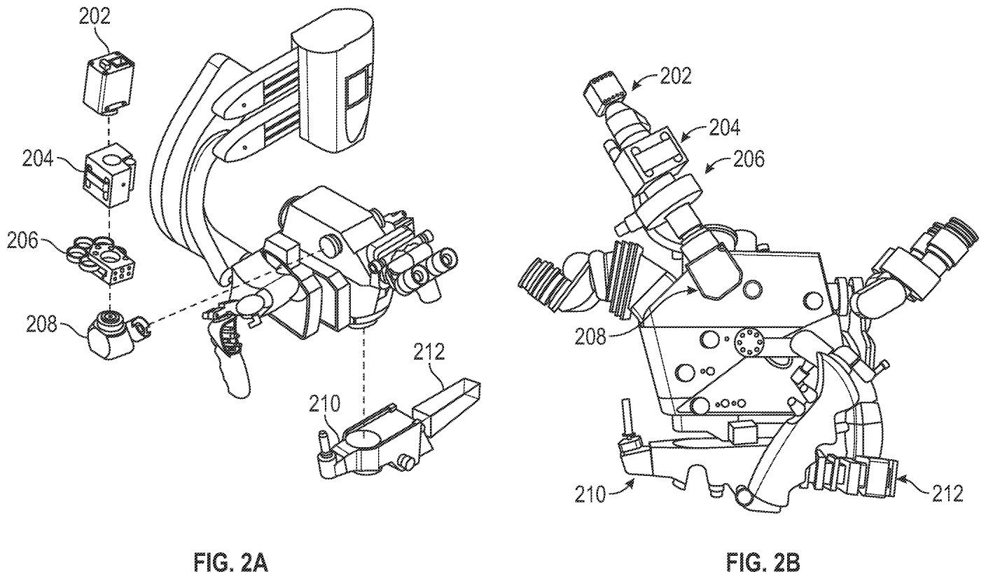

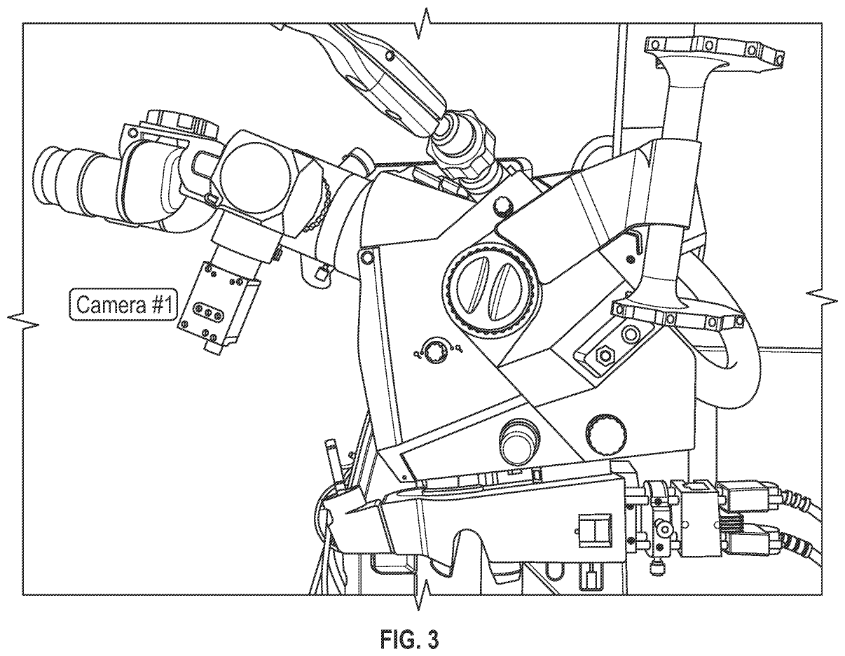

Referring now to FIGS. 2A, 2B, and 3, diagrams of various configurations of system 100 are shown, according to some implementations. In particular, FIGS. 2A and 2B illustrate an example configuration of system 100 attached to a microscope. FIG. 3 shows an example implementation of system 100 as described herein. It should be appreciated that, during testing, system 100 was found not to interfere with the sterile draping or normal operation of the microscope. In this example, a λ=785 nm laser diode 212—e.g., first light source 102—with a maximum output power of 300 mW was attached to an add-on laser adapter 210 (e.g., MM6 Micromanipulator, Carl Zeiss Meditec Inc., Oberkochen, Germany). It is possible for nm laser diode 212 to be attached externally, as in this example, or integrated internal to the microscope. Laser adapter 210 was mounted to the bottom of the microscope such that a steering mirror (not shown) directed the light downward toward the surgeon's field of view. The beam size was approximately 2 cm at a working distance of 35 cm. The maximum irradiance was 0.10 W/cm², well below the American National Standards Institute (ANSI) limit of 0.3 W/cm² for skin at 785 nm.³²

Back-scattered laser light was directed to an NIR-enhanced CMOS camera 202 (e.g., Basler AG, Ahrensburg, Germany) mounted on the side observer port on the same side as the craniotomy via a camera adapter 208. This enabled an observer to participate during the study. The pixel area was slightly cropped during acquisition to capture only pixels over brain tissue. A filter wheel 206 (e.g., CFW6, Thorlabs Inc.) and polarizer 204 (e.g., LPNIR100, Thorlabs Inc.) were positioned between camera adapter 208 and camera 202. Filter wheel 206 held various neutral density filters for controlling power output of nm laser diode 212. Polarizer 204 was integrated into a motorized rotation mount (e.g., RSC-100, Pacific Laser Equipment Inc., Santa Ana, California, USA) to reduce specular reflections. A band-pass filter (FF01-788/3-25, Semrock Inc., Rochester, New York, USA) was added in front of NIR-enhanced CMOS camera 202 (not shown) to enable simultaneous LSCI acquisition during illumination of indocyanine green and to block non-laser light and to avoid interference of normal white light illumination throughout each procedure.

It should be appreciated that, in the example of FIGS. 2A and 2B, laser diode 212 may be the same as or equivalent to first light source 102; polarizer 204 and filter wheel 206 may, together, form first optics 104; and camera 202 may the same as or equivalent to first camera 110. In some implementations, one or more of camera 202, polarizer 204, and filter wheel 206 can be coupled to the microscope via C-mount adapters or cage rods. In some implementations, second light source 106, second optics 108, and second camera 112 are integrated into the surgical microscope and are therefore not shown. Also not shown in FIGS. 2A and 2B, camera 202 and second camera 112 may be connected to an external computer.

### Computing Device

Referring now to FIG. 4, a computing device 400 for implementing the image analysis techniques described herein is shown, according to some implementations. In some implementations, remote computing device 114 is the same as or is functionally equivalent to computing device 400. Accordingly, it will be appreciated that FIG. 4 may be considered a detailed block diagram of remote computing device 114. More generally, computing device 400 is a computing device that is configured to obtain laser speckle images and/or white light images from first camera 110 and/or second camera 112 in order to generate and display blood flow visualizations.

Computing device 400 is shown to include a processing circuit 402 that includes a processor 404 and a memory 410. Processor 404 can be a general-purpose processor, an application specific integrated circuit (ASIC), one or more field programmable gate arrays (FPGAs), a group of processing components, or other suitable electronic processing structures. In some embodiments, processor 404 is configured to execute program code stored on memory 410 to cause computing device 400 to perform one or more operations, as described below in greater detail. It will be appreciated that, in embodiments where computing device 400 is part of another computing device, the components of computing device 400 may be shared with, or the same as, the host device. For example, if computing device 400 is implemented via a server, then computing device 400 may utilize the processing circuit, processor(s), and/or memory of the server to perform the functions described herein.

Memory 410 can include one or more devices (e.g., memory units, memory devices, storage devices, etc.) for storing data and/or computer code for completing and/or facilitating the various processes described in the present disclosure. In some embodiments, memory 410 includes tangible (e.g., non-transitory), computer-readable media that stores code or instructions executable by processor 404. Tangible, computer-readable media refers to any physical media that is capable of providing data that causes computing device 400 to operate in a particular fashion. Example tangible, computer-readable media may include, but is not limited to, volatile media, non-volatile media, removable media and non-removable media implemented in any method or technology for storage of information such as computer readable instructions, data structures, program modules or other data. Accordingly, memory 410 can include random access memory (RAM), read-only memory (ROM), hard drive storage, temporary storage, non-volatile memory, flash memory, optical memory, or any other suitable memory for storing software objects and/or computer instructions. Memory 410 can include database components, object code components, script components, or any other type of information structure for supporting the various activities and information structures described in the present disclosure. Memory 410 can be communicably connected to processor 404, such as via processing circuit 402, and can include computer code for executing (e.g., by processor 404) one or more processes described herein.

While shown as individual components, it will be appreciated that processor 404 and/or memory 410 can be implemented using a variety of different types and quantities of processors and memory. For example, processor 404 may represent a single processing device or multiple processing devices. Similarly, memory 410 may represent a single memory device or multiple memory devices. Additionally, in some embodiments, computing device 400 may be implemented within a single computing device (e.g., one server, one housing, etc.). In other embodiments, computing device 400 may be distributed across multiple servers or computers (e.g., that can exist in distributed locations). For example, computing device 400 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 410 is shown to include an image analyzer 412 which processes image data obtained from one or both of first camera 110 and second camera 112. In some implementations, image data is received from first camera 110 and/or second camera 112 and stored in a database 414 for later evaluation. “Image data,” as described herein, generally includes one or both of the laser speckle images captured by first camera 110 and the white light images captured by second camera 112. In some implementations, computing device 400 is in direct communication with first camera 110 and thus receives laser speckle images of a target area (e.g., target 120) of a subject directly from first camera 110. Similarly, computing device 400 may be in direct communication with second camera 112 to receive the white light images. In some implementations, one or both of the laser speckle images captured by first camera 110 and the white light images captured by second camera 112 are received indirectly, e.g., through another computing device.

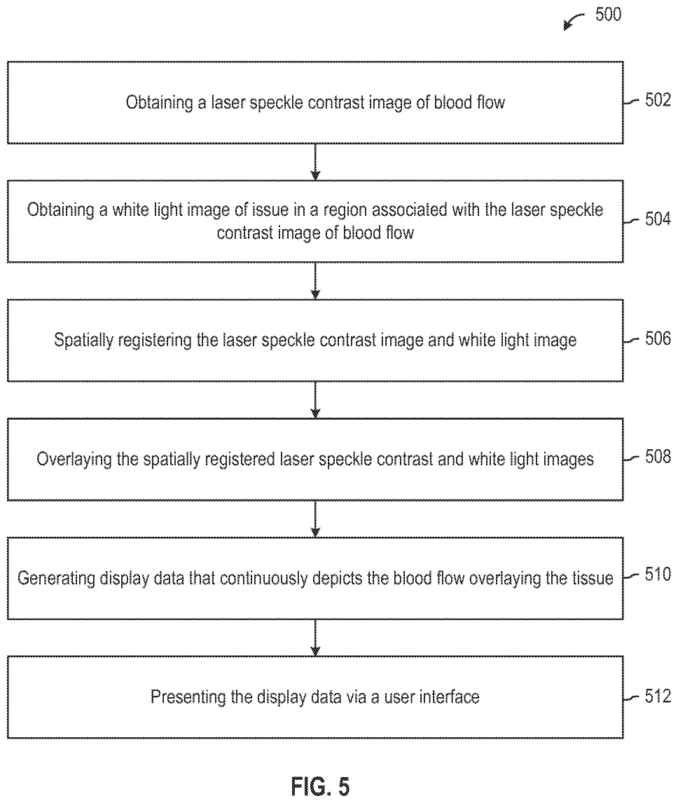

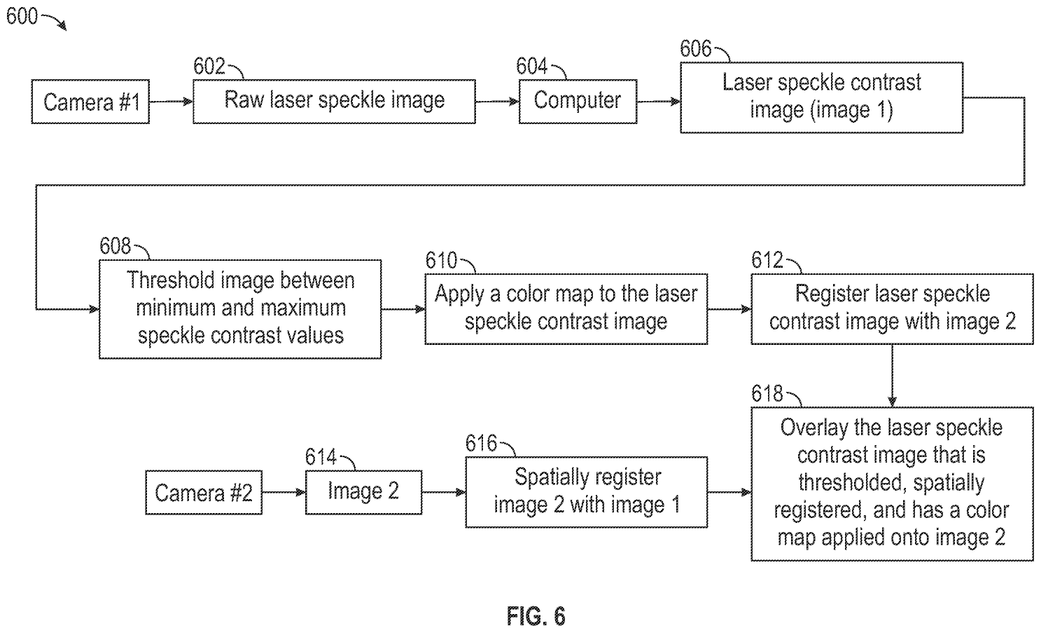

Image analyzer 412 is generally configured to implement the image analysis techniques described here, e.g., with respect to FIGS. 5 and 6, below. Generally, image analyzer 412 obtains both laser speckle images and white light images of a target area of a subject and performs various post-processing techniques. As mentioned above, laser speckle images generally capture blood flow in a subject while white light images generally capture an anatomical structure of the subject in a region associated with the laser speckle images of the blood flow. After receiving a raw laser speckle image-as a still image, a series of still images, or as a video feed-image analyzer 412 may derive a laser speckle contrast image or “LSCI image.” In some implementations, image analyzer 412 derived the laser speckle contrast image(s) using sliding pixel windows. In some implementations, image analyzer 412 applies thresholding to the laser speckle contrast image, as described in detail below. In some implementations, image analyzer 412 applies a color map to the laser speckle contrast image.

In some implementations, image analyzer 412 is configured to register the laser speckle contrast image with the white light image and/or vice-versa. In some such implementations, the laser speckle contrast image and white light images are spatially registered with one another, e.g., using an affine transformation. In some implementations, the laser speckle contrast image and white light images are spatially registered using a look-up table that maps pixels of the laser speckle contrast images and the white light images to one another. Subsequently, the co-registered laser speckle contrast image can be overlaid on the white light image. In some implementations, the co-registered and overlaid laser speckle contrast and white light images are used to generate display data that continuously depicts the blood flow overlaying the tissue. In some implementations, image analyzer 412 generates and then presents the display data via a user interface 422, described below.

Computing device 400 is also shown to include a communications interface 420 that facilitates communications between computing device 400 and any external components or devices. For example, communications interface 420 can provide means for transmitting data to, or receiving data from, first camera 110 and/or second camera 112. Accordingly, communications interface 420 can be or can include a wired or wireless communications interface (e.g., jacks, antennas, transmitters, receivers, transceivers, wire terminals, etc.) for conducting data communications, or a combination of wired and/or wireless communication interfaces. In some embodiments, communications via communications interface 420 are direct (e.g., local wired or wireless communications) or via a network (e.g., a WAN, the Internet, a cellular network, etc.). For example, communications interface 420 may include one or more Ethernet ports for communicably coupling computing device 400 to a network (e.g., the Internet). In another example, communications interface 420 can include a Wi-Fi transceiver for communicating via a wireless communications network. In yet another example, communications interface 420 may include cellular or mobile phone communications transceivers.

In some implementations, computing device 400 is communicably coupled to user interface 422 via communications interface 420 (e.g., via a wired connection). Generally, user interface 422 is an electronic device that allows a user to interact with computing device 400, e.g., by presenting data and/or receiving user inputs. To this point, user interface 422 generally includes at least a display (e.g., a screen) for displaying data. For example, user interface 422 may include an LED or LCD screen for displaying data (e.g., graphics, text, video, etc.). User interface 422 may, in particular, be configured to display at least the blood flow visualization data (e.g., video) generated by image analyzer 412. In some implementations, user interface 422 also includes at least one user input device for receiving user inputs. For example, user interface 422 may include one or more of a keyboard, a mouse, a number pad, arrow keys, buttons, and the like. In some implementations, user interface 422 is a separate computing device from computing device 400 that includes its own processor and/or memory. User interface 422 can include, for example, a mobile phone, an electronic tablet, a laptop or desktop computer, etc. In some implementations, rather than being directly connected to computing device 400, user interface 422 may be connected wirelessly (e.g., via the Internet).

### Image Analysis

As mentioned above, raw laser speckle images collected by first light source 102/camera 202 are processed by remote computing device 114 to calculate the spatial speckle contrast value (K) within a sliding moving window (e.g., a 7×7 pixel window) according to the equation

$$K = \frac{{σ}_{s}}{〈 I 〉} ,$$

where σₛ is the spatial standard deviation and [symbol] I [symbol] is the average intensity within the region. In some implementations, the image captured by first camera 110/camera 202 (e.g., “Image 1”) can be spatially transformed to the image captured by second camera 112 (e.g., “Image 2”). In other implementations, the image captured by second camera 112 (e.g., “Image 2”) can be spatially transformed to the image captured by first light source 102/camera 202 (e.g., “Image 1”). In yet other implementations, no spatial transform is applied. One example of a spatial transformation that could be applied is an affine transformation.

For relative blood flow measurements, speckle contrast was converted into correlation time (τ_{c}) by evaluating the average decay time of the speckle electric field autocorrelation function for which we assumed unity for the instrumentation factor. The speckle correlation time τ_{c} is a more quantitative measure of blood flow; the inverse correlation time (ICT=1/τ_{c}) is commonly used as a metric for blood flow in vessels or perfusion in parenchyma. For displaying ICT time course data, the first five seconds of data was used as a normalization factor to more easily visualize the change in flow relative to the baseline value. To overlay LSCI with images from the built-in microscope white light camera, the video output of the surgical microscope system was recorded continuously. LSCI images, white light reflectance images, and ICGA images were spatially co-registered by applying an affine transformation to all corresponding images.

Referring now to FIG. 5, a flow diagram of a process 500 for visualizing blood flow is shown, according to some implementations. In some implementations, process 500 is implemented by remote computing device 114, e.g., computer device 400, as described herein. It will be appreciated that certain steps of process 500 may be optional and, in some implementations, process 500 may be implemented using less than all of the steps. It will also be appreciated that the order of steps shown in FIG. 5 is not intended to be limiting.

At step 502, a laser speckle contrast image (e.g., LSCI image) of blood flow is obtained. In some implementations, the laser speckle contrast image is derived from a raw laser speckle image captured by first camera 110. In some such implementations, the laser speckle contrast image is derived from raw laser speckle image using a sliding pixel window. As mentioned above, in some implementations, spatial speckle contrast value (K) is calculated from the raw laser speckle image within a sliding moving window (e.g., a 7×7 pixel window) according to the equation

$$K = \frac{{σ}_{s}}{〈 I 〉} ,$$

Where σₛ is the spatial standard deviation and [symbol] I [symbol] is the average intensity within the region. In some implementations, as described below with respect to FIG. 6, thresholding is applied to the laser speckle contrast image. In some implementations, as also described below with respect to FIG. 6, a color map is applied to the laser speckle contrast image.

At step 504, a white light image of tissue in a region of a subject associated with the blood flow is obtained. In some implementations, the white light image is captured by second camera 112. As described herein, a “white light” image refers to an image of a target area (e.g., tissue) that is illuminated with a substantially white light. Generally, the white light image captures an anatomical structure of a subject in a region associated with the laser speckle contrast image of the blood flow. In some implementations, “tissue” refers to vasculature or brain tissue.

At step 506, the laser speckle contrast image and the white light image are spatially registered with one another (e.g., co-registered). In some implementations, the laser speckle contrast image and the white light image are spatially registered using an affine transformation. In some implementations, the laser speckle contrast image and the white light image are spatially registered using a look-up table that maps pixels of the laser speckle contrast images and the white light images to one another. In some implementations, spatially registering the laser speckle contrast and white light images includes creating a lookup table based on a spatial transformation used to register the laser speckle contrast and white light images.

At step 508, the spatially registered laser speckle contrast image is overlaid on the white light image. In some implementations, overlaying the spatially registered laser speckle contrast and white light images includes mapping respective pixels from the laser speckle contrast image to respective pixels from the white light image using the lookup table created at step 506. In some implementations, overlaying the spatially registered laser speckle contrast and white light images includes contrast stretching the laser speckle contrast image, mapping the laser speckle contrast image to an n-bit color map, and/or performing a weighted sum with the white light image.

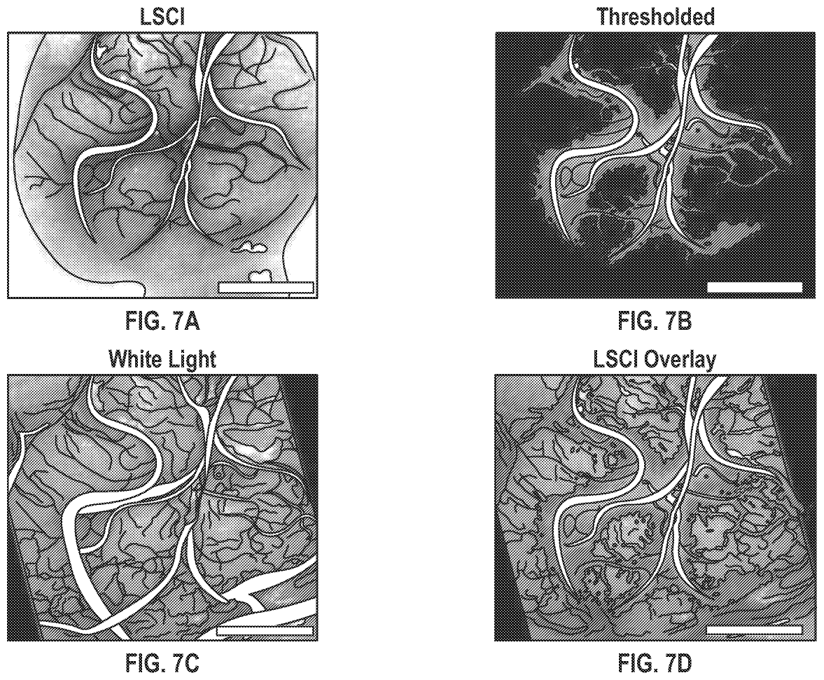

At step 510, display data is generated that continuously depicts blood flow (e.g., the laser speckle contrast image) overlaid on the tissue. Generally, the display data includes the spatially registered and overlaid laser speckle contrast and white light images. In some implementations, the display data includes a still image of the blood flow (e.g., from the laser speckle contrast image) overlaid on the tissue (e.g., from the white light image). In some implementations, the display data includes a series of images and/or video showing the blood flow over the tissue. An example of the display data is shown in FIG. 7D.

At step 512, the display data is presented via a user interface. In particular, the display data may be presented in real or near-real time to a user (e.g., a physician) via the user interface. In this way, the user can see (“visualize”) blood flow through/under the tissue in real or near-real time. An example of the display data is shown in FIG. 7D.

Referring now to FIG. 6, a flow diagram of a process 600 for visualizing blood flow is shown, according to some implementations. In some implementations, process 600 is implemented by remote computing device 114, e.g., computer device 400, as described herein. It will be appreciated that certain steps of process 600 may be optional and, in some implementations, process 600 may be implemented using less than all of the steps. It will also be appreciated that the order of steps shown in FIG. 6 is not intended to be limiting.

At step 602, first camera 110 captures a raw laser speckle image of a target area of a subject. In some implementations, the target area of the subject is illuminated via first light source 102, which may be a laser or another device that produces coherent light, as discussed above. In some implementations, the light from first light source 102 is passed through various filters and/or other optics, e.g., first optics 104. Generally, first light source 102 is configured to illuminate the target area to visualize blood flow. In some implementations, the light is reflected off of the target and back scattered light is filtered or reduced using a first filter that is configured to pass the wavelength or wavelength range output by first light source 102 while blocking wavelengths output by second light source 106. In some implementations, the filter is configured to enable simultaneous LSCI acquisition during illumination of indocyanine green and to block non-laser light. In some implementations, the back scattered light is polarized with a polarizing filter. In some implementations, first camera 110 captures the raw laser speckle image after filtering and polarizing to reduce backscatter.

At step 604, the raw laser speckle image is transmitted to remote computing device 114 or another computing device for post-processing.

At step 606, computing device 400 (e.g., remote computing device 114) derives a laser speckle contrast image or “LSCI image” from the raw laser speckle image. In some implementations, the laser speckle contrast images are based on sliding pixel windows. However, in other implementations, the images may not be acquired or processed based on sliding pixel windows. In some implementations, the laser speckle contrast images are spatial laser speckle contrast images. Thus, various implementations may be based on various neighborhood schemes. For example, in some implementations, the laser contrast speckle images may be derived based on temporal speckle contrast analysis or spatiotemporal laser speckle analyses. For non-spatial speckle contrast analysis, in some implantations, pseudo-color is applied to the LSCI images.

At step 608, computing device 400 (e.g., remote computing device 114) thresholds the LSCI image between minimum and maximum speckle contrast values. In some implementations, the laser speckle contrast images have laser speckle contrast values between a predetermined minimum speckle contrast value that is greater than zero and a predetermined maximum speckle contrast value. In some implementations, if the minimum speckle contrast value for thresholding is zero, no values will be discarded (with regard to a low threshold) since the theoretical minimum value for speckle contrast values is zero. However, in other implementations, the minimum value is greater than zero, in which case the system may discard the values below the selected minimum. This differs from transparency. In some implementations, transparency is applied only to values between the chosen minimum and maximum speckle contrast values and may refer to the amount of transparency to apply when the speckle contrast values are overlaid on the white light image of the tissue For example, 0% transparency would mean that the speckle contrast values are not visible and only the tissue is visible, whereas 100% transparency would mean the speckle contrast values are visible but not the tissue.

At step 610, computing device 400 (e.g., remote computing device 114) applies a color map to the LSCI image. In some implementations, the color map is an 8-bit color map; however, the color map may be any n-bit color map.

At step 612, the thresholded and/or color mapped LSCI image is registered with a second image (e.g., a white light image) captured by second camera 112 as described below. In some implementations, the LSCI image and second image are spatially registered using an affine transformation. In some implementations, the LSCI image and second image are spatially registered using a look-up table that maps pixels of the laser speckle contrast images and the white light images to one another.

In conjunction with the processing of the first image (e.g., the raw laser speckle image), at step 614, second camera 112 captures a second, white light image of the target area of the subject. As mentioned above, the white light may be emitted by second light source 106, through second optics 108, and onto the target area of the subject. In some implementations, the white light that is reflected from the tissue is filtered using a filter that is configured to pass the one or more wavelengths between 350 nm and 850 nm. In some implementations, the reflected white light is polarized using a polarizing filter.

As described herein, the “target area” of a subject is generally an area of tissue. “Tissue” may include any portion of the body and may be internal (e.g., brain surface) or external (e.g., skin or eye). It should be appreciated that, in some implementations, a single camera may be used to record the laser speckle contrast image and the white light images. This can be accomplished by interleaving the acquisition of laser speckle and white light images. For instance, the laser speckle image may be acquired with first optics 104 in front of a single camera, and then first optics 104 is switched with second optics 108 and the white light images is recorded on the single camera. During this interleaving process, light source 102 and second light source 106 can illuminate the tissue continuously or controlled such that first light source 102 illuminates during the capture of the laser speckle image, and second light source 106 illuminates during the capture of the white light image.

Subsequently, the second image may be transmitted to remote computing device 114 for additional processing. At step 616, remote computing device 114 spatially registers the second image with the first LSCI image. In some implementations, the LSCI image and second image are spatially registered using an affine transformation. In some implementations, the LSCI image and second image are spatially registered using on a look-up table that maps pixels of the laser speckle contrast images and the white light images to one another.

At step 618, the spatially registered, thresholded, and color-mapped LSCI image is overlayed onto the second image. In this manner, remote computing device 114 can generate a respective continuous stream of red, green, blue (RGB) images that depict the blood flow overlaying the tissue. In some implementations, a “continuous stream of images” is defined as at least one frame/image per second. In some implementations, a “continuous stream of images” is video that is greater than 0.5 seconds in duration and/or is continuous. However, in other implementations the duration may be 10 seconds, 30 seconds, 60 seconds, or more.

To this point, it should be appreciated that, in some implementations, process 600 may be continuously repeated to generate a real-time or near real-time video feed that overlays the LSCI images onto the white light images in order to visualize blood flow it the context of the subject's anatomy. In some implantations, as described above, the overlayed LSCI and white light images/video is presented via a user interface, e.g., in real-time or near real-time. As used herein, “real time” is defined in a clinical sense in that the medical provider using the visualization system may visualize blood flow (e.g., pulses) in the video stream simultaneously with the blood flow as the flow is progressing in the patient. In some implementations, real time is at least five frames per second; however either the LSCI image or white light image may be acquired slower than five frames per second even though the overlay image is displayed at five frames per second or higher. In some implementations, the video stream includes a field of view that is greater than 5 mm×5 mm. In some implementations, the video stream includes RBG images that record cessation of blood flow due to surgical intervention. For instance, the LSCI images can be acquired continuously so a surgeon may immediately see if her or his actions have resulted in desired blocking (e.g., clipping) of blood flow.

In some implementations, in response to the laser speckle contrast images having laser speckle contrast values between a predetermined minimum speckle contrast value and a predetermined maximum speckle contrast value, the video stream includes blood flow that is greater than a minimum blood flow that is greater than or equal to zero and that is less than a maximum blood flow. In some implementations, in response to the laser speckle contrast images having laser speckle contrast values between a predetermined minimum speckle contrast value and a predetermined maximum speckle contrast value, the video stream does not include blood flow that is less than the minimum blood flow or that is more than the maximum blood flow. In some implementations, in response to the laser speckle contrast images having laser speckle contrast values between a predetermined minimum speckle contrast value and a predetermined maximum speckle contrast value, the video stream includes blood flow that is greater than a minimum blood flow that is greater than zero and that is less than a maximum blood flow. In some implementations, in response to the laser speckle contrast images having laser speckle contrast values between a predetermined minimum speckle contrast value and a predetermined maximum speckle contrast value, the video stream does not include blood flow that is less than the minimum blood flow or that is more than the maximum blood flow.

In some implementations, a third camera is used to capture fluorescence images, e.g., at the same time as the laser speckle and white light images are captured. In some implementations, the third camera is a NIR camera. Fluorescence imaging includes, for example, ICGA. In some implementations, laser speckle contrast imaging and additional imaging is simultaneously performed, wherein the laser speckle contrast images are based on the laser speckle contrast imaging and the additional imaging includes at least one of doppler imaging, ultrasound imaging, percutaneous transfemoral DSA, or combinations thereof. In some implementations, a single camera may be used to record the fluorescence images and the raw laser speckle images by interleaving the image acquisitions for laser speckle and fluorescence. In some implementations, a single camera could be used to perform the laser speckle contrast imaging, fluorescence imaging, and white light imaging as mentioned above.

In some implementations, although not shown in FIG. 6, process 600 can further include spatially registering the laser speckle contrast images, the white light images, and fluorescence images with one another. In some implementations, process 600 can further include simultaneously performing multi-spectral reflectance imaging (MSRI) and laser speckle contrast imaging, wherein the laser speckle contrast images are based on the laser speckle contrast imaging. In some implementations, process 600 can further include simultaneously performing hyper-spectral imaging and laser speckle contrast imaging, wherein the laser speckle contrast images are based on the laser speckle contrast imaging. In some implementations, prior to capturing laser speckle and/or white light images, a first field of view of first camera 110 is aligned with a second field of view of second camera 112. In some implementations, a first trajectory of first light source 102 is aligned with the second field of view.

FIGS. 7A-7D illustrate an example application of process 600, in which an example LSCI image is thresholded and overlaid on an example visible white light reflectance image. First, the LSCI image is acquired. An example LCSI image is shown in FIG. 7A. Next, a threshold is applied to the LSCI image such that only speckle contrast values corresponding to flow values within a certain range remain (i.e., high flow in vessels). The threshold is applied to both the LSCI image and the raw camera intensity pixels. By thresholding the raw camera intensity pixels to include only areas where there is adequate illumination, the LSCI image has less artifacts due to poor illumination. By thresholding the LSCI image between minimum and maximum speckle contrast values, the contrast of the LSCI image is enhanced for visualization such that regions of certain blood flow are optimized for mapping on to color look up table. In addition to thresholding, a 2D Gaussian blur filter is applied to smooth out the LSCI image. An example thresholded image is shown in FIG. 7B.

Next, laser speckle contrast values within the threshold bounds are mapped onto a color map. In some implementations, the color map is configured/set by a user. Example color maps may be grayscale, reverse grayscale, jet or reverse jet, parula or reverse parula, or the like. During this step, the LSCI image is also spatially registered with the white light image such that the pixels are aligned in the final overlay image. An example white light image is shown in FIG. 7C. Generally, spatially registering the LSCI and white light images is referenced to herein as “co-registering.” The spatial alignment is performed by a geometric transform that maps the LSCI onto the white light image, such as an affine transformation. After applying a threshold and colormap, the LSCI image is merged with the white light image. For this step, the transparence of the thresholded and registered LSCI image can be adjusted to produce different visualization effects. In some implementations, the thresholded and registered LSCI image is fully visible on top of the white light image. Alternatively, the thresholded and registered LSCI image may be partially transparent to show the anatomical structure underneath the thresholded and registered LSCI image. An example LCSI overlay image is shown in FIG. 7D.

As discussed above, the processing of LSCI frames by thresholding the raw intensity and speckle contrast values, registering the thresholded LSCI image with the white light image, and overlaying the thresholded and registered LSCI image onto the white light image to create the overlay image can be performed by remote computing device 114 or any other suitable computing device. In some implementations, LSCI and white light images are captured and/or processes continuously (e.g., at video rate) and can be displayed continuously to a neurosurgeon or other user, e.g., on a monitor in real-time.

Pseudocode for computer-implemented image analysis (e.g., process 500 and/or process 600) is provided below, using raw laser speckle image (I), speckle contrast image (K), white light image (W), low-light intensity threshold (t₁), Gaussian blur standard deviation (c), lookup table mapping pixels from K(x,y) to W(x,y) based on spatial transform calculated during image registration (LUT), minimum and maximum speckle contrast values to display (Kₘᵢₙ and Kₘₐₓ), minimum and maximum transparency of speckle contrast overlay (αₘᵢₙ and αₘₐₓ), speckle contrast value below which transparency is set to αₘₐₓ(Kₐ), 256×3 matrix defining a colormap where each row defines an RGB color (CMAP). The method output includes an overlay image (O) merging speckle contrast and white light images into single RGB image.

| | |
|---|---|
| 1. | If I(x, y) ≤ t1 |
| 2. | Let K(x, y) = 0 |
| 3. | Let K = blur(K, σ), where blur( ) is a two-dimensional Gaussian blur |
| | filter |
| 4. | For (x, y) in K |
| 5. | Let (x′, y′) = LUT(x, y) |
| 6. | Let K(x′, y′) = K(x, y) |
| 7. | For (x, y) in K′ |
| 8. | Let scale = (K′(x, y) − Kmin)/(Kmax − Kmin) |
| 9. | Let index = int(255 × scale + 0.5) such that {index\|0 ≤ index ≤ |
| | 255} |
| 10. | Let α = K′(x, y) × ( αₘₐₓ − αₘᵢₙ) + αₘₐₓ − K_{α} × (αₘₐₓ − |
| | αₘᵢₙ)/(K_{α} − Kₘₐₓ |
| 11. | Let O(x, y, :) = (1 − α) × W(x, y, :) + α × CMAP(index, :) |

As shown, a threshold is applied to K based on the values of I. Next, a two-dimensional (2D) Gaussian blur filter is applied to smooth out the LSCI image. Then, laser speckle contrast values K(x,y) are mapped onto a coordinate space W(x,y) that is aligned with the white light image calculated by a lookup table. Then, the laser speckle values are thresholded and scaled, mapped to an 8-bit colormap, and assigned a transparency in relation to the white light image.

### Experimental Results

To test system 100 and the corresponding methods, as described herein, experimental surgeries were formed on five different subjects. A summary of patient details is provided below in Table 1.

TABLE 1
Patient Details

| | | | Aneurysm | | # of | Intraop/ | |
|---|---|---|---|---|---|---|---|
| | | Age | Location/ | Preop | Times | Postop | Clinical |
| Patient | Gender | Range | Size | Imaging | ICGA | Imaging | Outcome |
| 1 | F | 37-47 | Right SCA | CTA | 1 | Intraop DSA | Transient |
| | | | 5 mm | DSA | | Postop CTA | CN III palsy |
| | | | | | | | resolved |
| 2 | F | 59-69 | Right ICA | CTA | 2 | Intraop DSA | No |
| | | | 11 mm | DSA | | | complication |
| 3 | F | 73-83 | Left MCA | MRI | 1 | Intraop DSA | No |
| | | | 11 mm | DSA | | | complication |
| 4 | F | 52-62 | Right MCA | MRA | 2 | Intraop DSA | Transient |
| | | | and ACOM | DSA | | | cognitive |
| | | | | | | | dysfunction |
| | | | | | | | resolved |

ACOM = anterior communicating artery; CN = cranial nerve; CTA = computed tomography angiography; DSA = digital subtraction angiography; ICA = internal carotid artery; ICGA = indocyanine green angiography; MCA = middle cerebral artery; MRA = magnetic resonance angiography; SCA = superior cerebellar artery.

Prior to surgery, the field of view of the camera used for LSCI (e.g., first camera 110) was co-aligned and centered with the built-in microscope camera (e.g., second camera 112). Additionally, the laser beam was centered with the built-in microscope camera field of view. After the craniotomy was performed, the microscope was positioned over the patient at the discretion of the neurosurgeon. LSCI could be performed at any time when the microscope was positioned over the patient by turning on the laser illumination. LSCI did not disturb the workflow of the neurosurgeon and was performed at numerous critical times throughout the surgery. The neurosurgical co-investigator performed a majority of the surgeries using the surgical microscope oculars and could observe the LSCI data in real-time on a monitor mounted next to the surgical microscope which displayed the overlaid blood flow images.

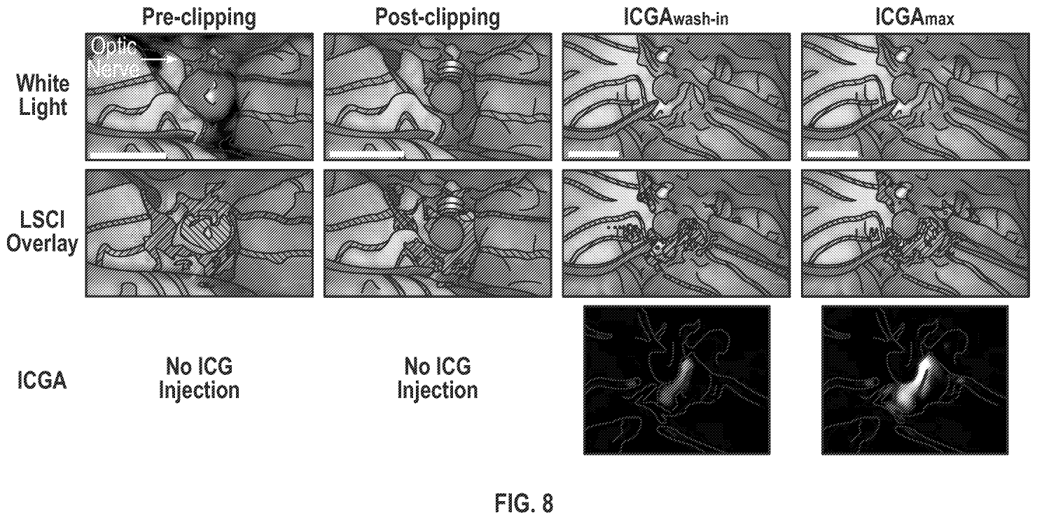

Referring now to FIG. 8, images from single time points pre-clipping and post-clipping of the aneurysm, and during ICGA for “Patient 2,” are shown. In particular, FIG. 8 shows images acquired from “Patient 2” before aneurysm clipping (pre-clipping), immediately after aneurysm clipping (post-clipping), and during ICGA at the wash-in of the dye (ICGA_{wash-in}) and at maximum fluorescence signal (ICGAₘₐₓ). Visible light images were acquired from the built-in microscope white light camera (white light); laser speckle contrast imaging (LSCI) images were acquired by an NIR-enhanced CMOS camera adapted to the microscope; and ICGA images were acquired by the built-in microscope NIR camera. LSCI overlay images were created by thresholding LSCI images and overlaying them onto the white light image with pseudo-color. Scale bars are one cm. FIG. 8 shows a montage of the white light and LSCI overlay images before, during, and after the clipping procedure. FIG. 8 also shows a montage of the white light, LSCI overlay, and ICGA images during the injection of the ICG dye.

In particular, white light and LSCI images were acquired throughout the duration of the aneurysm clipping, and ICGA images are only available during the injection of the indocyanine green dye after the aneurysm clip was placed. In this surgery, a temporary clip was placed on the patient's carotid artery in the neck after the pre-clipping images but before the aneurysm clipping. The pre-clipping LSCI images in FIG. 8 show there is high flow within the aneurysm prior to clipping, and that LSCI has the spatial resolution to visualize flow on the small vessels on the optic nerve (identified by arrow on pre-clipping white light image in FIG. 8). LSCI was used to visualize the filling of the aneurysm during cardiac cycle and the pulsatile motion of the flow within the aneurysm.

This was particularly evident when the temporary clip was placed on the patient's carotid artery in the neck causing a reduction of flow in the aneurysm. An advantage of having the LSCI instrumentation integrated into the microscope, as opposed to a stand-alone device, is that images can be acquired during operation of the microscope without interrupting the surgical workflow. This advantage is illustrated when the clip was placed on the aneurysm and the LSCI blood flow map immediately showed there is a cessation of flow in the aneurysm. This is similarly illustrated in the post-clipping LSCI image in FIG. 8. Approximately five minutes after the clipping, ICGA is used to confirm successful aneurysmal obliteration and confirm patency in surrounding vessels. In FIG. 8, the ICGA image during maximum fluorescent signal (ICGAₘₐₓ) reveals cessation of flow in the aneurysm.

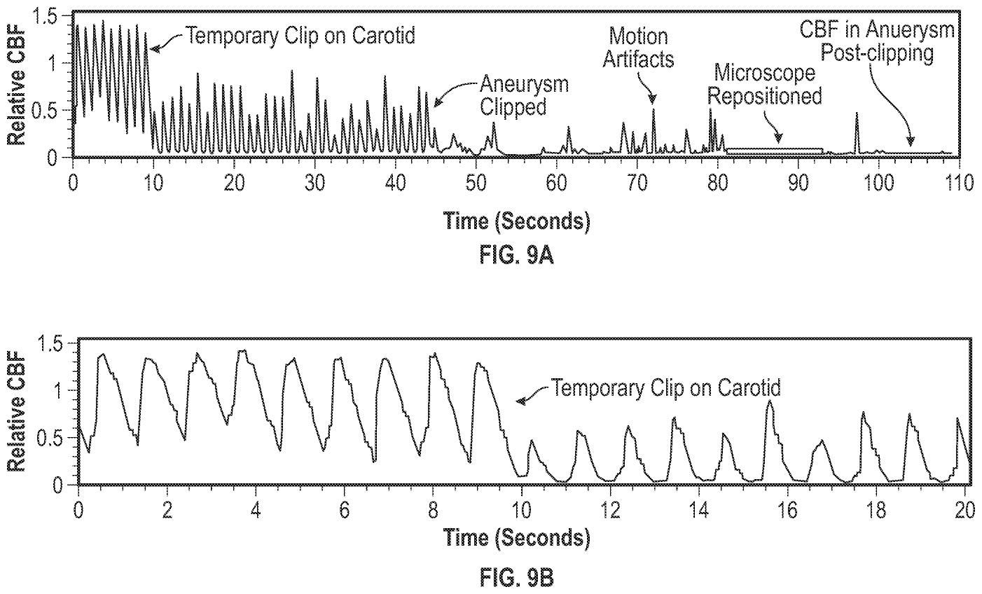

Referring now to FIGS. 9A and 9B, example graphs of relative CBF during a clipping procedure are shown, according to some implementations. In particular, FIGS. 9A and 9B show time courses of relative CBF within the aneurysm from “Patient 2” during the clipping procedure for 110 seconds (FIG. 9A) and zoomed in to the first 20 seconds (FIG. 9B). The relative CBF is normalized to the first five seconds of the data. The pulsatile nature of the flow in the aneurysm is clearly visible before and after the temporary clip is placed on the carotid artery. The reduction of CBF after the carotid temporary clip is immediately evident. Post-clipping cessation of flow is also clear. All the transients after aneurysm clipping (t=45s) are motion artifacts.

In this regard, the flow dynamics within the aneurysm were quantified in FIGS. 9A and 9B. The pulsatile flow in the aneurysm is visible before and after the temporary clip is placed on the carotid artery in the neck. There is about a 70% reduction in average CBF after the temporary clip is placed on the carotid and the pulsatile flow within the aneurysm is clearly visible with nulls of <5% and variable peaks of 50-80% of baseline. After the clip is placed on the aneurysm, there is a significant reduction in flow and complete disappearance of pulsatile flow. The transient spikes after the clip placement are due to motion artifacts from the surgeon mechanically pushing on the aneurysm. After the microscope is repositioned, it is obvious that the CBF within the aneurysm is absent and is within the lower limit of single exposure LSCI measurements (approximately 4% of the initial CBF).

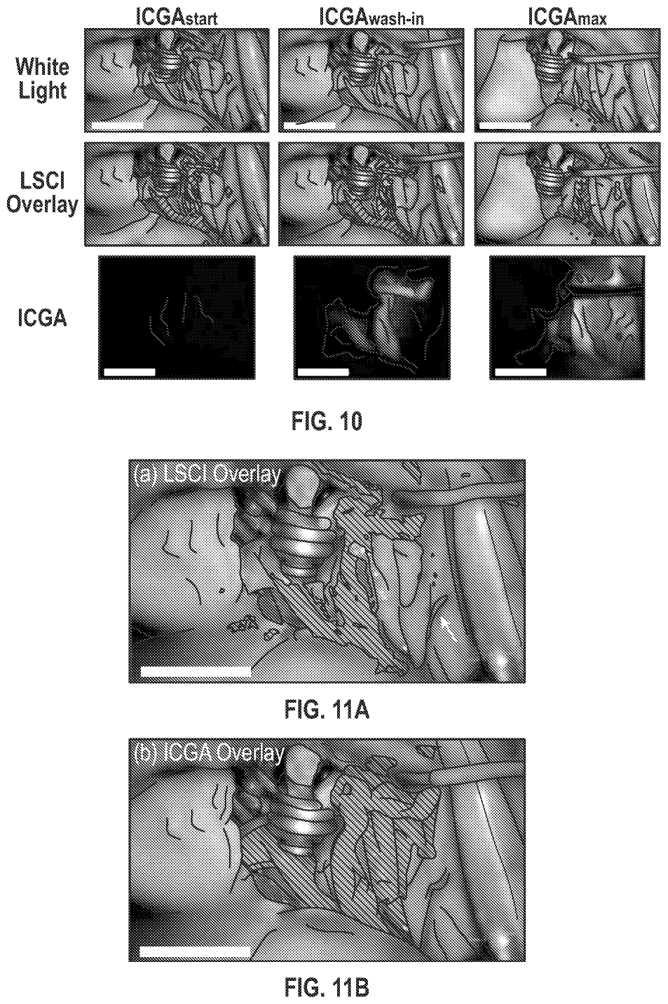

Referring now to FIG. 10, multiple example images captured from a patient during ICGA are shown, according to some implementations. In particular, FIG. 10 includes images acquired from “Patient 4” during ICGA at the start of the injection (ICGAₛₜₐᵣₜ), wash-in of the dye (ICG_{wash-in}), and at maximum fluorescence signal (ICGAₘₐₓ). FIG. 10 shows a montage of the white light, ICGA, and LSCI blood flow images during the entire ICGA procedure for which green pseudo-color is used for the LSCI overlay. Visible light images were acquired from the built-in microscope white light camera (white light); laser speckle contrast imaging (LSCI) images were acquired by an NIR-enhanced CMOS camera adapted to the microscope; and ICGA images were acquired by the built-in microscope NIR camera. LSCI overlay images were created by thresholding LSCI images and overlaying them onto the white light image. Scale bars are one cm.

Referring now to FIGS. 11A and 11B, example images that compare LSCI overlay with ICGA overlay are shown, according to some implementations. In particular, FIGS. 11A and 11B provide a comparison of (FIG. 11A) laser speckle contrast imaging (LSCI) overlay with (FIG. 11B) indocyanine green angiography (ICGA) overlay from “Patient 4.” The images were created by overlaying the LSCI data and ICGA data, respectively, onto the built-in microscope white light camera and applying green-pseudo-color. The arrow highlights LSCI's ability to detect blood flow in sidewall vessels. Scale bars are one cm.

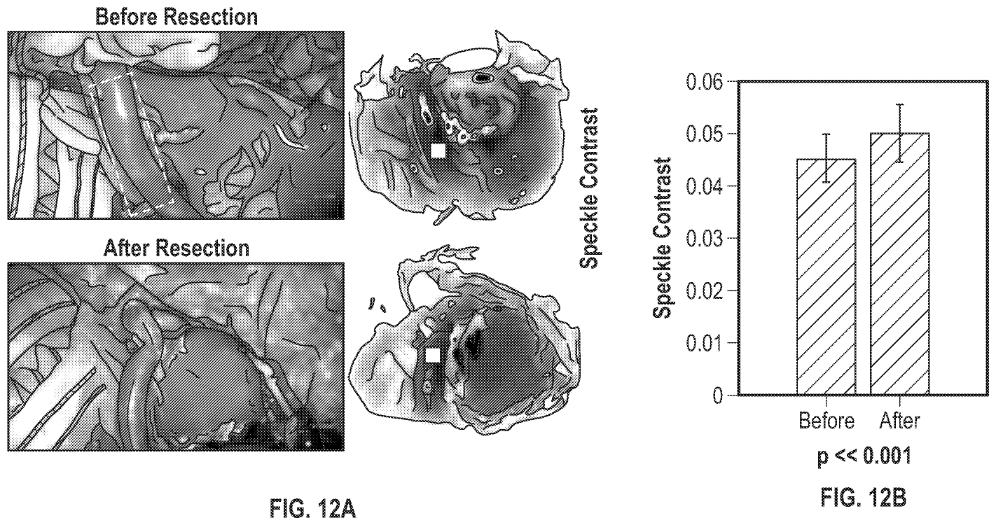

Referring now to FIGS. 12A and 12B, images captured during an arteriovenous malformation (AVM) resection from “Patient 5” are shown, according to various implementations. In particular, FIG. 12A includes multiple example images of an arteriovenous malformation (AVM) resection, according to some implementations. FIG. 12B is an example graph of averaged speckle contrast values before and after the AVM resection, according to some implementations. In FIG. 12A, a white light image showing the draining vein (outlined) of the AVM before the AVM is resected is shown. In addition, FIG. 12A includes a LSCI visualization in grayscale taken at same time as the white light image. A box on the vein represents the region of interest plotted in FIG. 12B. FIG. 12A also includes a light image showing the draining vein after the AVM is resected and a grayscale LSCI visualization taken at same time as the white light image. A box in these images defines a region of interest plotted in FIG. 12B. Accordingly, FIG. 12B is a plot of the averaged speckle contrast values from the orange highlighted boxes before and after the AVM resection.

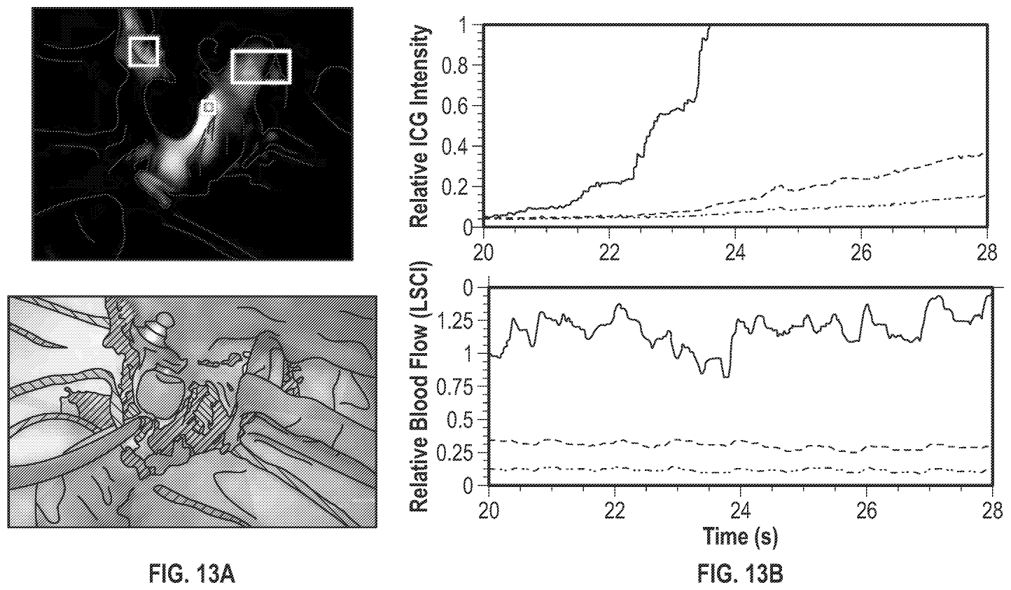

Referring now to FIGS. 13A and 13B, a time-course of the fluorescence intensity change during bolus administration of ICG and LSCI measures of relative blood flow for the same regions of interest are shown, according to various implementations. In particular, FIG. 13A includes multiple example images illustrating a fluorescence intensity change during bolus administration of indocyanine green (ICG) and the LSCI measures of relative blood flow for the same regions of interest, according to some implementations. FIG. 13B is an example graph of fluorescence intensity change during bolus administration of ICG and the LSCI measures of relative blood flow for the same regions of interest, according to some implementations. Three regions of interest, for which the time-course of the fluorescence intensity is shown in FIG. 13B, are highlighted in FIG. 13A. FIG. 13A includes, on bottom, an LSCI overlay image from the same time as the ICGA image on the top of FIG. 13A. The three regions of interest selected from the LSCI overlay displayed in FIG. 13B are the same regions as highlighted in FIG. 13A. FIG. 13B quantifies a time-course of the changes in the ICG fluorescence intensity (top) and relative blood flow measured by LSCI (bottom) during bolus administration of ICG.

### Discussion of Results

FIGS. 12A and 12B demonstrate the ability of LSCI for long term monitoring of flow in the draining vein of the AVM (outlined in FIG. 12A) for “Patient 5.” FIG. 12A shows the white light image and grayscale speckle contrast image before the AVM resection, respectively, taken at the same time point; the draining vessel is arterialized before the resection due to the AVM. In addition, FIG. 12A shows the white light image and grayscale speckle contrast image after the resection, respectively, taken at the same time point one hour and 35 minutes. FIG. 12B plots the averaged speckle contrast values in the orange highlighted regions of interest in FIG. 12A demonstrating that the flow in the draining vein after the AVM resection is lower than before the resection, suggesting that flow is no longer bypassing capillary networks through the AVM.

Results of the current study show that LSCI can be used to continuously monitor CBF in real-time. FIG. 8, as discussed above, and implementations addressed herein demonstrate LSCI's ability to continuously monitor CBF during critical parts of neurosurgical procedures when integrated into the surgical microscope and overlaid onto the surgeon's view of the surgical field. LSCI reveals high flow in the aneurysm before the aneurysm clipping, and immediately after clipping, the aneurysm flow ceases while the surrounding vessels maintain perfusion. ICGA is then used to confirm patency in the surrounding vessels but requires administration of a dye. This demonstrates that LSCI is complementary to ICGA; both can be used to determine perfusion in vessels within the surgical field of view.

Results shown in FIGS. 9A and 9B demonstrate that LSCI enables quantification of flow in the aneurysm relative to a baseline value. LSCI can detect the pulsatile flow profile within the aneurysm before the aneurysm is clipped. After clipping, LSCI shows there is a >96% reduction of flow in the aneurysm relative to the initial flow and no pulsatile flow. The uncertainty in relative flow for LSCI measurements may be approximately 5% since LSCI is sensitive to any form of motion within the tissue; thus, the aneurysm may be fully occluded and yet the LSCI signal will not reduce by 100% from baseline. It is expected that the LSCI signal would still be sensitive to Brownian motion of scattering particles within the aneurysm, however this signal will be significantly smaller than flow from red blood cells into the aneurysm. While further work is needed to determine the percentage reduction of flow for a surgeon to be confident the aneurysm is fully occluded, FIG. 10 offers preliminary evidence that a 96% reduction in flow in an aneurysm as measured by LSCI indicates successful aneurysmal obliteration.

The waveforms in FIGS. 9A and 9B depicting the CBF within the aneurysm match those measured with Doppler ultrasonography and a Doppler velocity wire. FIGS. 9A and 9B highlight that LSCI allows for continuous CBF measurements within an aneurysm throughout neurosurgery, and thus LSCI may be useful for improving our understanding of the hemodynamics in aneurysms and validating computational fluid dynamic models of the growth and rupture of aneurysms.

FIGS. 12A and 12B demonstrate the ability of LSCI for long term monitoring of flow in a surface vessel during AVM resection. FIG. 12A, in particular shows the surface vessel next to the AVM is initially arterialized and then flow is reduced after the AVM resection. This demonstration shows LSCI has the potential to quantify flow in feeding and draining vessels in real-time over the course of an AVM resection, which can take several hours, thus providing vital and actionable information to the surgeon on the success of the surgery. Future work will aim at establishing the repeatability of such flow measurements when the surgical environment changes.

FIGS. 13A and 13B further illustrate the complementary nature of information obtained with LSCI and ICGA when integrated into the surgical microscope. ICGA intensity is a more direct measure of cerebral blood volume (CBV) whereas LSCI is directly sensitive to motion, and therefore is a more direct measure of CBF. ICGA wash-in can also be used to identify feeding versus draining vessels in some surgical procedures and the temporal dynamics of the fluorescence intensity can be used to estimate CBF. FIGS. 13A and 13B show the time-course of the fluorescence intensity change during bolus administration of ICG along with the LSCI measures of relative blood flow for the same regions of interest. In this particular example the ICG fluorescence signal saturates in the largest vessel, but it is still clear that the rise time of the ICG signal is more rapid in this vessel than the two smaller vessels, indicating higher flow. The LSCI signals from the same regions reveal similar information, but in a different manner. The LSCI signals are steady state because they are direct measures of flow, which is relatively constant over the measurement period. However, the relative flow across each region of interest is evident from the steady state values of the speckle decorrelation times. Therefore, although LSCI is unable to quantify absolute CBF, it can be used to estimate relative CBF over time or across spatial regions.

Despite the differences between LSCI and ICGA, LSCI can also be used to create images that look very similar to ICGA images. Results comparing simultaneous LSCI and ICGA images shown in FIG. 11A-11B and implementations addressed herein demonstrate the complementary nature of information provided by LSCI during ICGA. The LSCI overlay image (FIG. 11A) and ICGA overlay image (FIG. 11B) show similar spatial information when rendered with similar green colormaps and overlaid onto the surgeon's view.

ICGA is better able to visualize flow in larger vessels due to ICGA using fluorescent dye as a contrast, whereas LSCI uses the inherent properties of the blood flow to scatter laser light. Conversely, LSCI has an advantage in visualizing flow in small vessels, as witnessed in FIG. 11 on the blood vessels in the side walls marked by the blue arrow; and in FIG. 8 for which LSCI shows CBF in small vessels supplying the optic nerve. ICGA also has the advantage of providing the directionality of flow during the wash-in of the dye.

One limitation of LSCI is that LSCI is sensitive to motion both from blood flow and the surgeon's mechanical force on the brain tissue. Thus, the LSCI overlay videos cannot differentiate between the two sources of motion. However, the motion induced by the surgeon can be avoided by the surgeon pausing for a moment and viewing the LSCI overlay while not pushing on the tissue.

Implementations addressed herein demonstrate the potential of LSCI to monitor CBF continuously during neurovascular surgery. The integration of the LSCI device into the surgical microscope enabled continuous CBF visualization and allowed for simultaneous acquisition of LSCI and ICGA. The time course of relative blood flow shown in FIGS. 9A, 9B and 11, and the LSCI overlays addressed herein demonstrate the ability of LSCI to monitor CBF continuously and in real-time. The sequence of simultaneous LSCI and ICGA images shown in FIGS. 8, 10, 11, and 13A suggest that LSCI and ICGA provide complementary information about CBF as both can be used to determine perfusion in a vessel.

Implementations suggest that LSCI can provide continuous and real-time CBF visualization without affecting the surgeon workflow or requiring a contrast agent, and thus is a promising tool for continuous CBF monitoring during surgery. By integrating the LSCI device into the surgical microscope, implementations perform LSCI at critical parts of neurovascular surgery and provide the surgeon with immediate actionable information on the success of the procedure. Implementations allow for simultaneous acquisition of LSCI and ICGA, demonstrating that LSCI and ICGA are complementary tools for visualizing CBF to aid surgical decision making.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

### EXAMPLE IMPLEMENTATIONS

Example 1. A blood flow visualization method comprising: [A] simultaneously illuminating: (a) blood flow with first light from a first light source, and (b) tissue with a second light from a second light source, wherein: (c) (i) the first light source is a coherent light source and the first light has a first wavelength, and (c) (ii) the second light source is a white light source and the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording white light images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than zero and a predetermined maximum speckle contrast value; [F] spatially registering the laser speckle contrast images and the white light images with one another; [G] in response to spatially registering the laser speckle contrast images with the white light images, overlaying the laser speckle contrast images with the white light images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 2. A blood flow visualization method comprising: [A] illuminating: (a) blood flow with first light from a first light source, and (b) tissue with a second light from a second light source, wherein: (c) (i) the first light includes a first wavelength, and (c) (ii) the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] spatially registering the laser speckle contrast images and the second camera's images with one another; [G] in response to spatially registering the laser speckle contrast images with the second camera's images, overlaying the laser speckle contrast images with the second camera's images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 3. A blood flow visualization method comprising: [A] simultaneously illuminating: (a) blood flow with first light from a first light source, and (b) tissue with a second light from a second light source, wherein: (c) (i) the first light source is a coherent light source and the first light has a first wavelength, and (c) (ii) the second light source is a white light source and the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording white light images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] spatially registering the laser speckle contrast images and the white light images with one another; [G] in response to spatially registering the laser speckle contrast images with the white light images, overlaying the laser speckle contrast images with the white light images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 4. A blood flow visualization method comprising: [A] illuminating: (a) blood flow with first light from a first light source, and (b) tissue with a second light from a second light source, wherein: (c) (i) the first light source is a coherent light source and the first light has a first wavelength, and (c) (ii) the second light source is a white light source and the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording white light images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] spatially registering the laser speckle contrast images and the white light images with one another; [G] in response to spatially registering the laser speckle contrast images with the white light images, overlaying the laser speckle contrast images with the white light images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 5. A blood flow visualization method comprising: [A] illuminating: (a) blood flow with first light from a first light source, and (b) tissue with a second light from a second light source, wherein: (c) (i) the first light includes a first wavelength, and (c) (ii) the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a camera; [D] in response to illuminating the tissue with the second light, recording non-raw laser speckle images with the camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] spatially registering the laser speckle contrast images and the non-raw laser speckle images with one another; [G] in response to spatially registering the laser speckle contrast images with the non-raw laser speckle images, overlaying the laser speckle contrast images with the non-raw laser speckle images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 6. A blood flow visualization method comprising: [A] illuminating: (a) blood flow with first light from a first light source, and (b) tissue with a second light from a second light source, wherein: (c) (i) the first light includes a first wavelength, and (c) (ii) the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording images with a second camera; [E] deriving laser speckle contrast images from the raw laser speckle images, wherein the laser speckle contrast images have laser speckle contrast values between a predetermined minimum speckle contrast value that is greater than or equal to zero and a predetermined maximum speckle contrast value; [F] spatially registering the laser speckle contrast images and the second camera's images with one another; [G] in response to spatially registering the laser speckle contrast images with the second camera's images, overlaying the laser speckle contrast images with the second camera's images to generate a respective continuous stream of red, green, blue (RGB) images that depict the blood flow overlaying the tissue; [H] outputting the continuous stream of the RBG images that depict the blood flow overlaying the tissue as a video stream, wherein the video stream is greater than 0.5 seconds in duration and is continuous.
Example 7. A blood flow visualization method comprising: [A] illuminating: (a) blood flow with first light from a first light source, and (b) tissue with a second light from a second light source, wherein: (c) (i) the first light includes a first wavelength, and (c) (ii) the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] spatially registering the laser speckle contrast images and the second camera's images with one another; [G] in response to spatially registering the laser speckle contrast images with the second camera's images, overlaying the laser speckle contrast images with the second camera's images to generate a stream of images that depict the blood flow overlaying the tissue; [H] outputting the stream of images that depict the blood flow overlaying the tissue as a video stream.
Example 8. A blood flow visualization method comprising: [A] illuminating: (a) blood flow with first light from a first light source, and (b) tissue with a second light from a second light source, wherein: (c) (i) the first light includes a first wavelength, and (c) (ii) the second light has one or more wavelengths between 350 nm and 850 nm; [B] in response to illuminating the blood flow with the first light, (a) filtering back scattered light from the blood flow with a first filter that is configured to pass the first wavelength, and (b) polarizing the back scattered light with a first polarizer; [C] in response to filtering and polarizing the back scattered light, recording raw laser speckle images with a first camera; [D] in response to illuminating the tissue with the second light, recording images with a second camera; [E] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than or equal to zero and a predetermined maximum speckle contrast value; [F] registering the laser speckle contrast images and the second camera's images with one another; [G] in response to registering the laser speckle contrast images with the second camera's images, overlaying the laser speckle contrast images with the second camera's images to generate red, green, blue (RGB) images that depict the blood flow overlaying the tissue; [H] outputting the RBG images that depict the blood flow overlaying the tissue.
Example 9. The method of any of examples 1-8, comprising outputting the video stream in real time with simultaneously illuminating the blood flow and the tissue.
Example 10. The method of any of examples 1-9, wherein the spatially registered laser speckle contrast images and white light images are based on affine transformations.
Example 11. The method of any of examples 1-10, wherein the second camera is included in one of a surgical microscope, an endoscope, an exoscope, and a robotic surgery platform. Example 12. The method of any of examples 1-11, wherein the video stream includes a field of view that is greater than 5 mm×5 mm.
Example 13. The method of any of examples 1-12, wherein: [A] in response to the laser speckle contrast images having laser speckle contrast values between a predetermined minimum speckle contrast value and a predetermined maximum speckle contrast value, the video stream includes blood flow that is greater than a minimum blood flow that is greater than or equal to zero and that is less than a maximum blood flow; [B] in response to the laser speckle contrast images having laser speckle contrast values between a predetermined minimum speckle contrast value and a predetermined maximum speckle contrast value, the video stream does not include blood flow that is less than the minimum blood flow or that is more than the maximum blood flow.
Example 14. The method of any of examples 1-12, wherein: [A] in response to the laser speckle contrast images having laser speckle contrast values between a predetermined minimum speckle contrast value and a predetermined maximum speckle contrast value, the video stream includes blood flow that is greater than a minimum blood flow that is greater than zero and that is less than a maximum blood flow; [B] in response to the laser speckle contrast images having laser speckle contrast values between a predetermined minimum speckle contrast value and a predetermined maximum speckle contrast value, the video stream does not include blood flow that is less than the minimum blood flow or that is more than the maximum blood flow.
Example 15. The method of any of examples 1-14, including, in response to illuminating the tissue with the second light, (a) filtering reflected white light that is reflected from the tissue with a second filter that is configured to pass the one or more wavelengths between 350 nm and 850 nm, and (b) polarizing the reflected white light reflected from the tissue with a second polarizer.
Example 16. The method of any of examples 1-15, wherein the first filter is configured to block the second light's one or more wavelengths between 350 nm and 850 nm. However, in other implementations, the first filter is configured to enable simultaneous LSCI acquisition during illumination of indocyanine green and to block non-laser light.
Example 17. The method of any of examples 1-16, wherein the first and second light sources are fixedly coupled to one another to move in tandem with another in response to manipulation by a user of the first and second light sources.
Example 18. The method of any of examples 1-17, comprising applying a color map to the laser speckle contrast images.
Example 19. The method of any of examples 1-18, comprising spatially registering the laser speckle contrast images and the white light images with one another based on a look-up table that maps pixels of the laser speckle contrast images and the white light images to one another.
Example 20. The method of any of examples 1-19, comprising: [A] separating the laser speckle contrast images that have laser speckle contrast values between the predetermined minimum speckle contrast value and the predetermined maximum speckle contrast value from the laser speckle contrast images that do not have laser speckle contrast values between the predetermined minimum speckle contrast value and the predetermined maximum speckle contrast value; [B] in response to separating the laser speckle contrast images, spatially registering the laser speckle contrast images and the white light images with one another.
Example 21. The method of any of examples 1-20, comprising in response to spatially registering the laser speckle contrast images and the white light images with one another, separating the laser speckle contrast images that have laser speckle contrast values between the predetermined minimum speckle contrast value and the predetermined maximum speckle contrast value from the laser speckle contrast images that do not have laser speckle contrast values between the predetermined minimum speckle contrast value and the predetermined maximum speckle contrast value.
Example 22. The method of any of examples 1-21, comprising simultaneously performing fluorescence imaging and laser speckle contrast imaging, wherein the laser speckle contrast images are based on the laser speckle contrast imaging.
Example 23. The method of example 22, further comprising simultaneously recording the raw laser speckle images with the first camera, the white light images with the second camera, and fluorescence images with a third camera. In some implementations, The third camera may include a NIR camera.
Example 24. The method of any of examples 1-23, comprising spatially registering the laser speckle contrast images, the white light images, and fluorescence images with one another.
Example 25. The method of any of examples 1-24, comprising simultaneously performing multi-spectral reflectance imaging (MSRI) and laser speckle contrast imaging, wherein the laser speckle contrast images are based on the laser speckle contrast imaging.
Example 26. The method of any of examples 1-25, comprising simultaneously performing hyper-spectral imaging and laser speckle contrast imaging, wherein the laser speckle contrast images are based on the laser speckle contrast imaging
Example 27. The method of any of examples 1-26, comprising: [A] aligning a first field of view of the first camera with a second field of view of the second camera; [B] aligning a first trajectory of the first light source with the second field of view.
Example 28. The method of any of examples 1-27, wherein the video stream includes RBG images that record cessation of blood flow due to surgical intervention. For instance, the LSCI images can be acquired continuously so a surgeon may immediately see if her or his actions have resulted in desired blocking (e.g., clipping) of blood flow.
Example 29. The method of any of examples 1-28, including determining a transparency level for the laser speckle contrast images having laser speckle contrast values between the predetermined minimum speckle contrast value and the predetermined maximum speckle contrast value
Example 30. The method of any of examples 1-29, wherein the laser speckle contrast images are spatial laser speckle contrast images.
Example 31. A blood flow visualization method comprising: [A] determining laser speckle contrast imaging (LSCI) images of blood flow; [B] apply a threshold to the LSCI images to separate first LSCI images from second LSCI images, wherein the first LSCI images correspond to higher blood flow levels than the second LSCI images; [C] apply pseudo-color to the first LSCI images; [D] overlaying the first LSCI images with white light images that were acquired simultaneously with speckle images that correspond to the first LSCI images.
Example 32. At least one non-transitory machine readable medium comprising a plurality of instructions that in response to being executed on a computing device, cause the computing device to carry out a method according to any one of examples 1-31.
Example 33. A processor-based system arranged to carry out a method according to any one of examples 1-31.
Example 34. An apparatus comprising means for performing any one of examples 1-31.
Example 35. A blood flow visualization system comprising: [A] first and second light sources; [B] a first filter and a first polarizer; [C] first and second cameras; and [D] at least one machine-readable medium having stored thereon data, which if used by at least one machine, causes the at least one machine to perform a method comprising: [D][i] simultaneously illuminating: (a) blood flow with first light from the first light source, and (b) tissue with a second light from the second light source, wherein: (c)(i) the first light source is a coherent light source and the first light has a first wavelength, and (c)(ii) the second light source is a white light source and the second light has one or more wavelengths between 350 nm and 850 nm; [D][ii] in response to illuminating the blood flow with the first light, (a) filtering back scattered light from the blood flow with a first filter that is configured to pass the first wavelength, and (b) polarizing the back scattered light with a first polarizer; [D][iii] in response to filtering and polarizing the back scattered light, recording raw laser speckle images with the first camera; [D][iv] in response to illuminating the tissue with the second light, recording white light images with the second camera; [D][v] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than zero and a predetermined maximum speckle contrast value; [D][vi] spatially registering the laser speckle contrast images and the white light images with one another; [D][vii] in response to spatially registering the laser speckle contrast images with the white light images, overlaying the laser speckle contrast images with the white light images to generate a respective continuous stream of red, green, blue (RGB) images that depict the blood flow overlaying the tissue; [D][viii] outputting the continuous stream of the RBG images that depict the blood flow overlaying the tissue as a video stream, wherein the video stream is greater than 0.5 seconds in duration and is continuous.
Example 36. A blood flow visualization system comprising: [A] first and second light sources, wherein: (a) the first light source is a coherent light source that emits a first light having a first wavelength, and (b) the second light source is a white light source that emits a second light having one or more wavelengths between 350 nm and 850 nm; [B] a first filter and a first polarizer; [C] first and second cameras; and [D] at least one machine-readable medium having stored thereon data, which if used by at least one machine, causes the at least one machine to perform a method comprising: [D][i] in response to illuminating blood flow with the first light and tissue with the second light, recording raw laser speckle images with the first camera, and white light images with the second camera; [D][ii] deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than zero and a predetermined maximum speckle contrast value; [D][iii] spatially registering the laser speckle contrast images and the white light images with one another; [D][iv] in response to spatially registering the laser speckle contrast images with the white light images, overlaying the laser speckle contrast images with the white light images to generate a respective continuous stream of red, green, blue (RGB) images that depict the blood flow overlaying the tissue; [D][v] outputting the continuous stream of the RBG images that depict the blood flow overlaying the tissue as a video stream, wherein the video stream is greater than 0.5 seconds in duration and is continuous.
Example 37. The system of example 35 or 36, wherein the at least one machine-readable medium has stored thereon data, which if used by the at the least one machine, causes the at least one machine to perform a method comprising outputting the video stream in real time with simultaneously illuminating the blood flow and the tissue.
Example 38. The system according to any of examples 35-37, wherein the spatially registered laser speckle contrast images and white light images are based on affine transformations.
Example 39. The system according to any of examples 35-38, wherein the second camera is included in one of a surgical microscope, an endoscope, an exoscope, and a robotic surgery platform.
Example 40. The system according to any of examples 35-39, wherein the video stream includes a field of view that is greater than 5 mm×5 mm.
Example 41. The system according to any of examples 35-40, wherein: [A] in response to the laser speckle contrast images having laser speckle contrast values between a predetermined minimum speckle contrast value and a predetermined maximum speckle contrast value, the video stream includes blood flow that is greater than a minimum blood flow that is greater than or equal to zero and that is less than a maximum blood flow; [B] in response to the laser speckle contrast images having laser speckle contrast values between a predetermined minimum speckle contrast value and a predetermined maximum speckle contrast value, the video stream does not include blood flow that is less than the minimum blood flow or that is more than the maximum blood flow.
Example 42. The system according to any of examples 35-41, comprising a second filter and a second polarizer; wherein the second filter is configured to pass the one or more wavelengths between 350 nm and 850 nm, and the second polarizer is configured to polarize the reflected white light reflected from the tissue with a second polarizer.
Example 43. The system according to any of examples 35-42, wherein the first filter is configured to block the second light's one or more wavelengths between 350 nm and 850 nm.
Example 44. The system according to any of examples 35-43, wherein the first and second light sources are fixedly coupled to one another to move in tandem with another in response to manipulation by a user of the first and second light sources.
Example 45. The system according to any of examples 35-44, wherein the at least one machine-readable medium has stored thereon data, which if used by the at the least one machine, causes the at least one machine to perform a method comprising applying a color map to the laser speckle contrast images.
Example 46. The system according to any of examples 35-45, wherein the at least one machine-readable medium has stored thereon data, which if used by the at the least one machine, causes the at least one machine to perform a method comprising spatially registering the laser speckle contrast images and the white light images with one another based on a look-up table that maps pixels of the laser speckle contrast images and the white light images to one another.
Example 47. The system according to any of examples 35-46, wherein the at least one machine-readable medium has stored thereon data, which if used by the at the least one machine, causes the at least one machine to perform a method comprising: [A] separating the laser speckle contrast images that have laser speckle contrast values between the predetermined minimum speckle contrast value and the predetermined maximum speckle contrast value from the laser speckle contrast images that do not have laser speckle contrast values between the predetermined minimum speckle contrast value and the predetermined maximum speckle contrast value; [B] in response to separating the laser speckle contrast images, spatially registering the laser speckle contrast images and the white light images with one another.
Example 48. The system according to any of examples 35-47, wherein the at least one machine-readable medium has stored thereon data, which if used by the at the least one machine, causes the at least one machine to perform a method comprising in response to spatially registering the laser speckle contrast images and the white light images with one another, separating the laser speckle contrast images that have laser speckle contrast values between the predetermined minimum speckle contrast value and the predetermined maximum speckle contrast value from the laser speckle contrast images that do not have laser speckle contrast values between the predetermined minimum speckle contrast value and the predetermined maximum speckle contrast value.
Example 49. The system according to any of examples 35-48, wherein the at least one machine-readable medium has stored thereon data, which if used by the at the least one machine, causes the at least one machine to perform a method comprising simultaneously performing fluorescence imaging and laser speckle contrast imaging, wherein the laser speckle contrast images are based on the laser speckle contrast imaging.
Example 50. The system according to example 49, wherein the at least one machine-readable medium has stored thereon data, which if used by the at the least one machine, causes the at least one machine to perform a method comprising simultaneously recording the raw laser speckle images with the first camera, the white light images with the second camera, and fluorescence images with a third camera.
Example 51. The system according to example 49 or 50, wherein the at least one machine-readable medium has stored thereon data, which if used by the at the least one machine, causes the at least one machine to perform a method comprising spatially registering the laser speckle contrast images, the white light images, and fluorescence images with one another. Example 52. The system according to any of examples 35-51, wherein the at least one machine-readable medium has stored thereon data, which if used by the at the least one machine, causes the at least one machine to perform a method comprising simultaneously performing multi-spectral reflectance imaging (MSRI) and laser speckle contrast imaging, wherein the laser speckle contrast images are based on the laser speckle contrast imaging.
Example 53. The system according to any of examples 35-52, wherein the at least one machine-readable medium has stored thereon data, which if used by the at the least one machine, causes the at least one machine to perform a method comprising simultaneously performing hyper-spectral imaging and laser speckle contrast imaging, wherein the laser speckle contrast images are based on the laser speckle contrast imaging
Example 54. The system according to any of examples 35-53, wherein the video stream includes RBG images that record cessation of blood flow due to surgical intervention.
Example 55. The system according to any of examples 35-54, wherein the at least one machine-readable medium has stored thereon data, which if used by the at the least one machine, causes the at least one machine to perform a method comprising determining a transparency level for the laser speckle contrast images having laser speckle contrast values between the predetermined minimum speckle contrast value and the predetermined maximum speckle contrast value
Example 56. The system according to any of examples 35-55, wherein the laser speckle contrast images are spatial laser speckle contrast images.
Example 57. At least one machine-readable medium having stored thereon data, which if used by at least one machine, causes the at least one machine to perform a method comprising: [A] determining laser speckle contrast imaging (LSCI) images of blood flow; [B] applying a threshold to the LSCI images to separate first LSCI images from second LSCI images, wherein the first LSCI images correspond to higher blood flow levels than the second LSCI images; [C] applying pseudo-color to the first LSCI images; [D] overlaying the first LSCI images with white light images that were acquired simultaneously with speckle images that correspond to the first LSCI images.
Example 58. A blood flow visualization method comprising: [A] receiving a laser speckle contrast imaging (LSCI) image of blood flow; [B] receiving a white light image of a tissue, the white light image capturing an anatomical structure; [C] registering the LSCI image and the white light image with one another; [D] overlaying the registered LSCI and white light images; and [E] generating display data that continuously depicts the blood flow overlaying the tissue, wherein the display data comprises the registered LSCI and white light images.
Example 59. The method of example 57 or 58, wherein the display data continuously depicts the blood flow overlaying the tissue in real-time during a surgical procedure.
Example 60. The method of any one of examples 57-59, further comprising displaying the display data on a display device.
Example 61. The method of any one of examples 57-60, wherein the tissue comprises vasculature.
Example 62. The method of any one of examples 57-61, wherein the tissue is brain tissue.
Example 63. The method of any one of examples 57-62, wherein the step of registering the LSCI image and the white light image with one another comprises creating a lookup table based on a spatial transformation used to register the LSCI and white light images.
Example 64. The method of any one of examples 57-63, wherein the step of overlaying the registered LSCI and white light images comprises mapping respective pixels from the LSCI image to respective pixels from the white light image using the lookup table.
Example 65. The method of any one of examples 57-64, wherein the step of overlaying the registered LSCI and white light images further comprises contrast stretching the LSCI image, mapping the LSCI image to an n-bit color map, and performing a weighted sum with the white light image.
Example 66. At least one non-transitory machine readable medium comprising a plurality of instructions that in response to being executed on a computing device, cause the computing device to carry out a method according to any one of examples 57-65.
Example 67. A system for blood flow visualization comprising: [A] first and second light sources, the first light source being configured to illuminate blood flow, and the second light source being configured to illuminate a tissue with white light; [B] first and second cameras, wherein the first camera is configured to record a raw laser speckle image of the blood flow, and the second camera is configured to record a white light image of the tissue; and [C] a computing device comprising a processor and a memory, the memory having stored thereon computer-executable instructions that, when executed by the processor, cause the processor to: [C][i] receive the raw laser speckle image of the blood flow; [C][ii] derive a laser speckle contrast imaging (LSCI) image; [C][iii] receive the white light image of the tissue, the white light image capturing an anatomical structure; [C][iv] register the LSCI image and the white light image with one another; [C][v] overlay the registered LSCI and white light images; and [C][vi] generate display data that continuously depicts the blood flow overlaying the tissue, wherein the display data comprises the registered LSCI and white light images.
Example 68. The system of example 67, wherein the system is a surgical microscope, an endoscope, an exoscope, or a robotic surgery platform.
Example 69. A method comprising: performing the blood flow visualization method according to any one of examples 57-65; and continuously monitoring the blood flow overlaying the tissue during a surgical procedure.
Example 70. The method of example 69, wherein the surgical procedure is a brain surgery. Example 71. The method of example 69, wherein the surgical procedure is a cerebral aneurysm clipping.
Example 72. The method of example 69, wherein the surgical procedure is an arteriovenous malformation (AVM) resection.
Example 73. A blood flow visualization system comprising: a first light source; a first filter and a first polarizer; a first camera; and at least one machine-readable medium having stored thereon data, which if used by at least one machine, causes the at least one machine to perform a method comprising: illuminate blood flow with first light from the first light source, wherein the first light source is a coherent light source and the first light has a first wavelength; in response to illuminating the blood flow with the first light, (a) filtering back scattered light from the blood flow with the first filter that is configured to pass the first wavelength, and (b) polarizing the back scattered light with the first polarizer; in response to filtering and polarizing the back scattered light, recording raw laser speckle images with the first camera; receiving white light images derived from a second camera; deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than zero and a predetermined maximum speckle contrast value; spatially registering the laser speckle contrast images and the white light images with one another; in response to spatially registering the laser speckle contrast images with the white light images, overlaying the laser speckle contrast images with the white light images to generate a respective continuous stream of red, green, blue (RGB) images that depict the blood flow overlaying the tissue; outputting the continuous stream of the RBG images that depict the blood flow overlaying the tissue as a video stream, wherein the video stream is greater than 0.5 seconds in duration and is continuous.
Example 74. A blood flow visualization system comprising: a first light source to illuminate blood flow with first light, wherein the first light source is a coherent light source and the first light has a first wavelength; a first filter and a first polarizer to respectively (a) filter back scattered light from the blood flow with the first filter that is configured to pass the first wavelength, and (b) polarize the back scattered light with the first polarizer; a first camera; and at least one machine-readable medium having stored thereon data, which if used by at least one machine, causes the at least one machine to perform a method comprising: in response to filtering and polarizing the back scattered light, recording raw laser speckle images with the first camera; receiving white light images derived from a second camera; deriving laser speckle contrast images from the raw laser speckle images, wherein: (a) the laser speckle contrast images are based on sliding pixel windows, and (b) the laser speckle contrast images have laser speckle contrast values between a predetermined minimum speckle contrast value that is greater than zero and a predetermined maximum speckle contrast value; spatially registering the laser speckle contrast images and the white light images with one another; in response to spatially registering the laser speckle contrast images with the white light images, overlaying the laser speckle contrast images with the white light images to generate a respective continuous stream of red, green, blue (RGB) images that depict the blood flow overlaying the tissue; outputting the continuous stream of the RBG images that depict the blood flow overlaying the tissue as a video stream, wherein the video stream is greater than 0.5 seconds in duration and is continuous.

1. A method for visualizing blood flow, the method comprising:
obtaining a laser speckle contrast imaging (LSCI) image of blood flow;
obtaining a white light image of a tissue, the white light image capturing an anatomical structure of a subject in a region associated with the LSCI image of the blood flow;
spatially registering the LSCI image and the white light image with one another;
overlaying the spatially registered LSCI and white light images; and
generating display data that continuously depicts the blood flow overlaying the tissue, wherein the display data comprises the spatially registered LSCI and white light images.

2. The method of claim 1, wherein the display data continuously depicts the blood flow overlaying the tissue in real-time during a surgical procedure.

3. The method of claim 1, further comprising displaying the display data on a user interface.

4. The method of claim 1, wherein the tissue comprises vasculature.

5. The method of claim 1, wherein the tissue is brain tissue.

6. The method of claim 1, wherein spatially registering the LSCI image and the white light image with one another comprises creating a lookup table based on a spatial transformation used to register the LSCI and white light images.

7. The method of claim 6, wherein overlaying the spatially registered LSCI and white light images comprises mapping respective pixels from the LSCI image to respective pixels from the white light image using the lookup table.

8. The method of claim 7, wherein overlaying the spatially registered LSCI and white light images further comprises contrast stretching the LSCI image, mapping the LSCI image to an n-bit color map, and performing a weighted sum with the white light image.

9. A system for blood flow visualization, the system comprising:
a first light source configured to illuminate blood flow at a target region of a subject;
a first camera configured to record a raw laser speckle image of the blood flow; and
a computing device comprising a processor and a memory, the memory having instructions stored thereon that, when executed by the processor, cause the computing device to:
obtain, via the first camera, the raw laser speckle image of the blood flow;
derive a laser speckle contrast imaging (LSCI) image from the raw laser speckle image;
obtain a white light image of tissue at the target region of the subject, the white light image capturing an anatomical structure;
spatially register the LSCI image and the white light image with one another;
overlay the spatially registered LSCI and white light images; and
generate display data that continuously depicts the blood flow overlaying the tissue, wherein the display data comprises the spatially registered LSCI and white light images.

10. The system of claim 9, wherein the system is a surgical microscope, an endoscope, an exoscope, a robotic surgery platform, a stand-alone imaging system, or system dedicated for blood flow imaging.

11. The system of claim 9, wherein the display data continuously depicts the blood flow overlaying the tissue in real-time during a surgical procedure.

12. The system of claim 9, wherein the instructions further cause the computing device to present the display data on a user interface.

13. The system of claim 9, wherein the tissue comprises vasculature.

14. The system of claim 9, wherein the tissue is brain tissue.

15. The system of claim 9, wherein spatially registering the LSCI image and the white light image with one another comprises creating a lookup table based on a spatial transformation used to register the LSCI and white light images.

16. The system of claim 15, wherein overlaying the spatially registered LSCI and white light images comprises mapping respective pixels from the LSCI image to respective pixels from the white light image using the lookup table.

17. The system of claim 16, wherein overlaying the spatially registered LSCI and white light images further comprises contrast stretching the LSCI image, mapping the LSCI image to an n-bit color map, and performing a weighted sum with the white light image.

18. A method for visualizing blood flow, the method comprising continuously:
capturing, using a first image capture device, a laser speckle contrast imaging (LSCI) image of blood flow in a subject;
capturing, using a second image capture device, a white light image of a tissue, the white light image capturing an anatomical structure;
co-registering the LSCI image and the white light image;
overlaying the co-registered LSCI and white light images; and
displaying, via a user interface, the overlayed and co-registered LSCI and white light images.

19. The method of claim 18, wherein the tissue comprises vasculature or brain tissue.

20. The method of claim 18, wherein co-registering the LSCI image and the white light image with one another comprises creating a lookup table based on a spatial transformation used to register the LSCI and white light images.
